(19) United States
(12) Patent Application Publication
(10) Pub. No.: US 2026/0077020 A1
(43) Pub. Date: Mar. 19, 2026

(54) ORAL PEPTIDE PHARMACEUTICAL COMPOSITION THAT FACILITATES ENHANCED ORAL ABSORPTION

(71) Applicants: Danielle Marie Le Roux, West Milford, NJ (US); Jayendrakumar Dasharathlal Patel, Robbinsville, NJ (US); Shwetaben Dasharathbhai Patel, Vijapur (IN)

(72) Inventors: Danielle Marie Le Roux, West Milford, NJ (US); Jayendrakumar Dasharathlal Patel, Robbinsville, NJ (US); Shwetaben Dasharathbhai Patel, Vijapur (IN)

(21) Appl. No.: 19/342,040

(22) Filed: Sep. 26, 2025

(51) Int. Cl. A61K 38/26; A61K 9/00; A61K 9/20

(52) U.S. Cl. CPC A61K 38/26; A61K 9/0053; A61K 9/2009; A61K 9/2013; A61K 9/2027; A61K 9/2054; A61K 9/2086

(57) ABSTRACT

The present invention relates to a pharmaceutical formulation that comprises a drug, an alkaline salt of an oral absorption promoter, an acid-neutralizing agent, and optionally one or more pharmaceutically acceptable excipients, wherein the pharmaceutical formulation releases the acid neutralizing agent prior to, concurrently with, faster than, slower than or after the release of the alkaline salt of the oral absorption promoter. A method for the administration of the pharmaceutical composition, a method for treating disease conditions using the pharmaceutical composition, and a method for the preparation of the pharmaceutical composition are also disclosed.

### FIELD OF THE INVENTION

The presently disclosed subject matter relates to an oral pharmaceutical composition that improves oral absorption of a drug contained therein, specifically when administered in a fasted stomach. More specifically, the present invention relates to a pharmaceutical formulation that comprises a drug, an alkaline salt of an oral absorption promoter, an acid-neutralizing agent, and optionally one or more pharmaceutically acceptable excipients, wherein the pharmaceutical formulation releases the acid neutralizing agent prior to, concurrently with, faster than, slower than or after the release of the alkaline salt of the oral absorption promoter. A method for the administration of the pharmaceutical composition, a method for treating a disease condition using the pharmaceutical composition, and a method for the preparation of the pharmaceutical composition are also disclosed.

### BACKGROUND OF THE INVENTION

One of the main challenges in oral delivery of proteins and peptides is the inability of these compounds to be readily transported across the membranes of the gastrointestinal tract. Further, a gastric enzyme, such as pepsin, exhibits its optimum or prominent activity at a pH of about less than 2 in the stomach, but lower activity at pH>5 [Piper D W, Fenton B H. pH stability and activity curves of pepsin with special reference to their clinical importance. Gut. 1965 October; 6 (5): 506-8. doi: 10.1136/gut.6.5.506. PMID: 4158734; PMCID: PMC1552331]. So, drug(s) that are predominantly released in the stomach to allow maximum absorption from the upper part of the gastrointestinal tract, may exhibit a lower therapeutic effect if the drug(s) are prone to degradation by at least one of the gastric enzymes, such as pepsin, and the acidic pH of the fasted gastric medium. Further, various drugs, such as peptides, have at least one serine protease cleavage site, i.e., the peptide comprises one or more amino acid residue(s) amenable or prone to cleavage by a serine protease, particularly an intestinal serine protease, such as trypsin, chymotrypsin, aminopeptidase, carboxypeptidase, elastase and/or dipeptidyl-4-peptidase) and the likes. In major regions of the intestinal tract, the pH of an intestinal fluid remains greater than about 6, and the intestinal serine proteases exhibit their activity at a pH of greater than about 6, with optimal activity at a pH of about 7.5. Therefore, the drugs prone to proteolytic degradation by at least one intestinal enzyme, such as intestinal serine protease, may exhibit lower pharmacological activity when exposed to the intestinal environment.

The alkaline salt of an oral absorption promoter, such as sodium N-(8-(2-hydroxybenzoyl)amino) caprylic acid (SNAC), has previously been shown to improve the bioavailability of drugs, such as orally administered peptides when administered in a fasted state stomach. For example, RYBELSUS tablet [(semaglutide) tablets 7 mg or 14 mg], comprising the SNAC improved absorption of Semaglutide when administered in the fasted state stomach; however, the variability in the Semaglutide exposure was found to be very high after a single dose, as some subjects had concentrations below the lower limit of quantification.

Therefore, there is still a need in the art for a method that facilitates enhanced oral absorption of drugs that are prone to gastric or proteolytic degradation. There is still a need in the art for further improvement of the bioavailability of oral administration of a composition of drugs, such as peptides and proteins, when it is formulated using the alkaline salt of the oral absorption promoter, such as sodium N-(8-(2-hydroxybenzoyl)amino) caprylic acid (SNAC).

### SUMMARY OF THE INVENTION

SNAC contained in the RYBELSUS tablet [(semaglutide) tablets contained 7 mg or 14 mg of Semaglutide and SNAC] improved the absorption of semaglutide when administered in the fasted state stomach; however, the variability in the Semaglutide exposure was found to be very high after a single dose, as some subjects had concentrations below the lower limit of quantification. The inventors of the published U.S. Pat. No. 11,759,501B2 found that the bioavailability of GLP-1 peptide, such as Semaglutide, from the different compositions comprising various granule designs of the SNAC can be predicted from the in vitro dissolution or solubilization behavior of the SNAC. The granules that offered an improved dissolution rate of the SNAC had a marked improvement in the bioavailability of GLP-1 peptide from the composition.

The present inventors surprisingly found that the dissolution properties of an alkaline salt of oral absorption promoter contained in the pharmaceutical formulation are greatly improved in an acidic medium (e.g., acidic medium having a pH of less than about 2, e.g., 35 mL of 0.1N HCl or mixture of 35 mL of 0.1N HCl+125 mL of Water or 150 mL of acidic media having pH less than about 2), provided that at least one acid-neutralizing agent contained in the pharmaceutical formulation is released in the acidic medium prior to, concurrently with, or faster than the release of the alkaline salt of the oral absorption promoter from the pharmaceutical formulation. The present inventors surprisingly found that the dissolution properties of an alkaline salt of oral absorption promoter contained in the pharmaceutical formulation are greatly improved in an acidic medium (e.g., acidic medium having a pH of less than about 2, e.g., 35 mL of 0.1N HCl or mixture of 35 mL of 0.1N HCl+125 mL of Water or 150 mL of acidic media having pH less than about 2), provided that at least one acid-neutralizing agent contained in the pharmaceutical formulation is released in the acidic medium slower than, or after the release of the alkaline salt of the oral absorption promoter.

The present inventors surprisingly found that the dissolution properties of an alkaline salt of oral absorption promoter contained in the pharmaceutical formulation are greatly improved in an acidic medium having pH of less than 2 (but pH of greater than about 1) (e.g., acidic medium having a pH of less than about 2, e.g., 35 mL of 0.1N HCl or mixture of 35 mL of 0.1N HCl+125 ml of Water or 150 mL of acidic media having pH less than about 2), provided that the said pharmaceutical formulation is comprised of at least one acid neutralizer in the amount that is at least sufficient to elevate the pH of the acidic medium to at least 2 or greater [preferably elevate the pH to at least about 2.5, such as at least about 3, at least about 4, at least about 5, and more preferably at least about 5.5 or higher but less than about 8], and releases the said at least one acid neutralizer in the acidic medium prior to, concurrently with, or faster than the release of the alkaline salt of the oral absorption promoter. The present inventors also surprisingly found that the dissolution property of an alkaline salt of oral absorption promoter contained in the pharmaceutical formulation is markedly improved in an acidic medium having pH of less than 2 (but pH of greater than about 1) (e.g., acidic medium having a pH of less than about 2, e.g., 35 mL of 0.1N HCl or mixture of 35 mL of 0.1N HCl+125 mL of Water or 150 mL of acidic media having pH less than about 2), provided that the said pharmaceutical formulation is comprised of at least one acid neutralizer in the amount that is at least sufficient to elevate the pH of the acidic medium to at least 2 or greater [preferably elevate the pH to at least about 2.5, such as at least about 3, at least about 4, at least about 5, and more preferably at least about 5.5 or higher but less than about 8], and releases the said at least one acid neutralizer in the acidic medium slower than or after the release of the alkaline salt of the oral absorption promoter.

The present inventor surprisingly found that the dissolution behavior of the alkaline salt of oral absorption promoter incorporated in the pharmaceutical formulation, such as SNAC, could be improved in an acidic medium having the pH of less than about 2 (but greater than about 1), such as in the fasted gastric medium when administered in the fasted stomach, which can subsequently greatly improve the oral bioavailability of the drug contained in the pharmaceutical formulation, provided that the said pharmaceutical formulation contained at least one acid-neutralizing agent in the amount that is at least sufficient to elevate the pH of the acidic medium to at least 2 or greater [preferably elevate the pH to at least about 2.5, such as at least about 3, at least about 4, at least about 5, and more preferably at least about 5.5 or higher but less than about 8] and it releases the acid neutralizing agent in the acidic medium prior to, concurrently with, faster than, slower than or after the release of the alkaline salt of the oral absorption promoter from the pharmaceutical formulation.

The present inventors found that improved dissolution behavior of the alkaline salt of oral absorption promoter, such as SNAC, in the acidic medium having the pH of less than about 2 (but >1), such as in the fasted gastric medium when administered in the fasted stomach, can significantly improve the oral bioavailability of the drug contained in the pharmaceutical formulation when it administered in the fasted state stomach.

In preferred embodiment, the present invention relates to a pharmaceutical formulation that comprises a drug, an alkaline salt of oral absorption promoter, an acid-neutralizing agent (alkalizing agent), and optionally one or more pharmaceutically acceptable excipients, wherein the pharmaceutical formulation releases the acid neutralizing agent prior to, concurrently with, faster than, slower than or after the release of the alkaline salt of the oral absorption promoter from.

In certain embodiment, the present invention relates to a pharmaceutical formulation that comprises a drug, 5-95% w/w of an alkaline salt of oral absorption promoter, an acid-neutralizing agent in the amount that is at least sufficient to elevate the pH of mixture of 35 mL of 0.1N HCl and 125 ml of water from less than about 2 to at least about 2.5 (such as 3, 3.5, 4, 4.5, 5, 5.5, 6, 6.5, 7, or 7.5, but less than about 10, such as less than about 9), and optionally one or more pharmaceutically acceptable excipients, wherein the said pharmaceutical formulation when subjected to in-vitro dissolution in the mixture of 35 mL of 0.1N HCl and 125 ml of water (dissolution medium), using USP type II/I apparatus (paddle/basket, respectively) or equivalent dissolution apparatus in design (such as using smaller size paddle/basket or using smaller size® agent prior to, concurrently with, faster than, slower than or after the release of the alkaline salt of the oral absorption promoter, which subsequently greatly improve the dissolution behavior of the alkaline salt of oral absorption promoter in the dissolution medium.

In certain embodiment, the present invention relates to a pharmaceutical formulation that facilitates the enhanced dissolution of an alkaline salt of oral absorption promoter contained therein, wherein the said pharmaceutical formulation comprises (1) a drug in amount of about 0.01-85%, such as 0.1-75%, 0.5-70%, 0.75-60%, 1-55%, 1.5-50% (w/w) of the total amount of the pharmaceutical formulation, (2) an alkaline salt of oral absorption promoter in amount of about 1-99%, such as 5-98%, 10-96%, 15-95%, 30-90%, 35-85% (w/w) of the total amount of the pharmaceutical formulation, (3) an acid neutralizer in an amount of about 1-75%, 2-65%, 2.5-50%, 3-40%, 3.5-35% (w/w) of the total amount of the pharmaceutical formulation or in an amount that is at least sufficient to elevate the pH of mixture of 35 mL of 0.1N HCl and 125 ml of water from less than about 2 to at least about 2.5 (such as 3, 3.5, 4, 4.5, 5, 5.5, 6, 6.5, 7, or 7.5, but less than about 10, such as less than about 9, preferably less than about 8), and (4) optionally one or more pharmaceutically acceptable excipients, wherein the said pharmaceutical formulation when subjected to in-vitro dissolution in the mixture of 35 mL of 0.1N HCl and 125 ml of water (dissolution medium), using USP type II/I apparatus (paddle/basket, respectively) or equivalent dissolution apparatus in design (such as using smaller size paddle/basket or using smaller size dissolution vessel) at 50/75/100 RPM and 37° C.±0.5° C. temperature, it releases the acid neutralizing agent prior to, concurrently with, faster than, slower than or after the release of the alkaline salt of the oral absorption promoter, which subsequently greatly improve the dissolution behavior of the alkaline salt of oral absorption promoter in the dissolution medium.

In certain embodiment, the present invention relates to a pharmaceutical formulation that facilitates the enhanced dissolution of an alkaline salt of oral absorption promoter contained therein, wherein the said pharmaceutical formulation comprises (1) a drug in amount of about 0.01-85%, such as 0.1-75%, 0.5-70%, 0.75-60%, 1-55%, 1.5-50% (w/w) of the total amount of the pharmaceutical formulation, (2) an alkaline salt of oral absorption promoter in amount of about 1-99%, such as 5-98%, 10-96%, 15-95%, 30-90%, 35-85% (w/w) of the total amount of the pharmaceutical formulation, (3) an acid neutralizer in an amount of about 1-75%, 2-65%, 2.5-50%, 3-40%, 3.5-35% (w/w) of the total amount of the pharmaceutical formulation or in an amount that is at least sufficient to elevate the pH of 35 mL or 160 mL of acidic medium from less than about 2 to at least about 2.5 (such as 3, 3.5, 4, 4.5, 5, 5.5, 6, 6.5, 7, or 7.5, but less than about 10, such as less than about 9, preferably less than about 8), and (4) optionally one or more pharmaceutically acceptable excipients, wherein the said pharmaceutical formulation when subjected to in-vitro release test in the 35 mL or 160 mL of acidic medium, it releases the acid neutralizing agent prior to, concurrently with, faster than, slower than or after the release of the alkaline salt of the oral absorption promoter, which subsequently greatly improve the dissolution behavior of the alkaline salt of oral absorption promoter in the 35 mL or 160 mL of acidic medium, thereby facilitating marked improvement in the release or dissolution of the alkaline salt of oral absorption promoter in the 35 mL or 160 mL of the acidic medium.

The present inventors surprising found that the sequence of the release of the acid neutralizer in compared to the release of the alkaline salt of oral absorption promoter or the drug (i.e., at least about 2%, at least about 5%, at least about 20%, at least about 25%, at least about 35%, at least about 50%, at least about 75%, at least about 85% (w/w) of acid neutralizer release prior to beginning of release of alkaline salt of oral absorption promoter or the drug (the minimum amount of release of the acid-neutralizing agent does not limit the scope of the present invention, as it depends on the total amount of acid-neutralizing agent used in the formulation, and its release rate can be adjusted to elevate the pH of the acidic medium to at least greater than about 2 or at least about 5), release of acid neutralizer concurrently with release of alkaline salt of oral absorption promoter or the drug, release of acid neutralizer after release of alkaline salt of oral absorption promoter but prior to or faster than the release of the drug, release of acid neutralizer faster than release of alkaline salt of oral absorption promoter or the drug {such as release faster in first 45 minutes, such as first 30 minutes, first 25 minutes, first 20 minutes, first 15 minutes, first 10 minutes, first 5 minutes}, release of acid neutralizer slower than release of alkaline salt of oral absorption promoter {such as release slower in first 45 minutes, such as first 30 minutes, first 25 minutes, first 20 minutes, first 15 minutes, first 10 minutes, first 5 minutes} but prior to or faster than the release of the drug etc.) from the pharmaceutical formulation had a marked effect on the release or dissolution behavior of the alkaline salt of oral absorption promoter in the 35 mL or 160 mL of the acidic medium having a pH of less than about 2 (such as in fasted state stomach condition). For example, the acid neutralizer that completely released prior to the beginning release of the alkaline salt of oral absorption promoter provided 72.94% release or dissolution of the alkaline salt of oral absorption promoter at 2 minutes in 160 mL of the acidic medium, the acid neutralizer that released concurrently with the release of the alkaline salt of oral absorption promoter provided 10.287% release or dissolution of the alkaline salt of oral absorption promoter at 2 minutes in 160 mL of the acidic medium, and the acid neutralizer that released after the complete release of alkaline salt of oral absorption promoter provided only 3.65% release or dissolution of the alkaline salt of oral absorption promoter at 2 minutes in 35 mL or 160 mL of the acidic medium, however, in all cases, the percentage release or dissolution of the alkaline salt of the oral absorption promoter at 2 minutes in 35 mL or 160 mL of the acidic medium is significantly enhanced compared to the absence of the acid neutralizer at 2 minutes, which was 0.013% (refer to the examples of the present application).

### DETAILED DESCRIPTION OF THE INVENTION

The terms used in this specification generally have their ordinary meanings in the art, within the context of the presently disclosed subject matter, and in the specific context where each term is used. Certain terms are discussed below, or elsewhere in the specification, to provide additional guidance to the practitioner in describing the compositions and methods of the presently disclosed subject matter and how to make and use them. To the extent a term used in a claim is not defined below, it should be given the broadest definition persons in the pertinent art have given that term as reflected in printed publications and issued patents at the time of filing.

As used in this specification, the use of the word “a” or “an” or “the” when used in conjunction with the term “comprising” in the claims and/or the specification can mean “one,” but it is also consistent with the meaning of “one or more,” “at least one,” and “one or more than one.” Thus, for example, “a drug” includes reference to one or more drugs, “an oral absorption enhancer” includes one or a mixture of two or more oral absorption enhancers, and the like. Still further, the terms “having,” “including,” “containing”, “comprising”, and “comprises” are interchangeable, and one of skill in the art is cognizant that these terms are open-ended terms.

As used in this specification, the term “pharmaceutical composition” or “pharmaceutical formulation” refers to an oral pharmaceutical dosage form formulated as any of a solid oral dosage form and/or a liquid oral dosage form for oral administration, preferably, peroral administration. In certain embodiments, the oral pharmaceutical dosage form is selected from any or a combination of single or multilayer tablet (coated or uncoated tablet), capsules (soft gelatin capsules, hard gelatin capsules, HPMC capsules, or HPMCP capsules, etc.), a capsule-in-capsule, tablet-in-capsule, lozenges, troches, ovules, solutions, emulsions, suspensions, syrups, elixirs, powders and granules for reconstitution, dispersible powders and granules, medicated gums, chewing tablets, effervescent tablets, multi-particulate dosage forms (multi-particulate filled in capsule, or compressed with one or more pharmaceutical additives to form tablet), and the likes. The term “multi-particulate” as used herein means a plurality of discrete particles, pellets, mini-tablets, mini-capsules, or combinations thereof. However, any or a combination of the oral pharmaceutical dosage form(s), as known to or appreciated by a person skilled in the art, can be utilized to serve its intended purpose, as laid in the present disclosure, without departing from the scope and spirit of the present disclosure.

As used in this specification, the terms “drug”, “active substance”, “active ingredient”, “active agent”, “drug substance”, “pharmacologically active agent”, “pharmacological agent”, “physiologically active agent”, “drug substance in the physiologically active form” or “pharmaceutically acceptable derivatives of the drug” are used interchangeably herein to refer to a chemical compound that induces a desired pharmacological effect or biological activity. The terms pharmaceutically acceptable derivatives of the drug substance mentioned herein include, but are not limited to, salts, ether, stereo-isomer solvates, polymorphs, hydrates, complexes with one or more molecules, complexes with one or more cationic or anionic ingredient, drug-ion exchange resin complex, prodrug, active metabolites, analogs, homologue, and the like. In some embodiments, a drug substance is present in acid or base form. The term drug substance is also meant to encompass the use of all such possible forms as well as their racemic and resolved forms thereof, and all tautomers as well. The term “racemic” refers to a mixture of equal parts of enantiomers. In some embodiment, the drug substance is present in powder form, in amorphous form, in crystalline form, in micronized form, in complexes with one or more molecules, in a combination of two or more active substances, or any combination thereof. Further, the term “pharmacologically”, “biologically”, “therapeutically”, or “chemically” effective amount (dose) of the drug refers to an amount of the drug that, when administered to a subject/patient to treat a disease, is sufficient to effect treatment for the disease. The “therapeutically effective amount” can vary depending on the drug, the disease and its severity, and the age, weight, etc., of the subject to be treated.

As used in this specification, the term “oral absorption enhancer”, “oral absorption promoter”, “oral permeation enhancer” or “oral permeability enhancer” are used interchangeably herein to refer to an agent or ingredient that, when co-administered with a drug in same oral pharmaceutical dosage form, enhances or improves the absorption or permeation of the drug, thereby improve the bioavailability of the drug in the blood. Generally, the term “bioavailability”, as used herein, refers to the fraction of an administered dose of the drug which reaches the systemic circulation unchanged. By definition, when a drug is administered intravenously, its bioavailability is 100%. However, when it is administered via other routes (such as orally), its bioavailability decreases due to incomplete absorption and/or first-pass metabolism. Knowledge about bioavailability is important when calculating dosages for non-intravenous routes of administration. The term “an alkaline salt of an oral absorption promoter” refers to the oral absorption promoter or a derivative of the oral absorption promoter that is neutralized using any alkali/alkaline material. Preferably, but not limited to, the salt is a lithium, sodium, potassium, calcium, magnesium, ammonium or alkanolammonium, triethylammonium or triethanolammonium salt; more preferably, the salt is sodium or potassium.

As used in this specification, the term “intestinal enzyme inhibitors” refers to an agent or substance that reduces the activity of at least one intestinal enzyme, thereby affords the protection, at least in part, to a drug from proteolytic degradation by the intestinal enzyme. The term “proteolytic degradation” refers to the degradation of the drug by one or more intestinal enzymes, i.e., a serine protease, such as trypsin, chymotrypsin, aminopeptidase, carboxypeptidase, elastase and/or dipeptidyl-4-peptidase) and the likes.

As used in this specification, the terms “fasted”, “fasted state”, “fasted stomach”, or “fasted state stomach” are used interchangeably herein to refer to abstention from food and sometimes drinking to keep the stomach empty for a certain time period. The term “administration or ingestion in a fasted state” refers to the ingestion of an oral pharmaceutical composition on the fasted stomach, preferably before taking a meal, more preferably at least 15 minutes before taking a meal, most preferably at least 30 minutes before taking a meal, particularly at least 60 minutes before taking a meal, and most particularly at least 120 minutes before taking a meal. In certain embodiments, “administration in the fasted state” also refers to the ingestion of the oral pharmaceutical composition on the fasted stomach at least 600 minutes before taking a meal.

As used in this specification, the term “fasted gastric degradation” refers to degradation of a drug by any or a combined effect of the gastric enzyme, such as pepsin, and the acidic pH of the fasted gastric medium (less than about 2). A gastric enzyme, such as pepsin, exhibits its optimum or prominent activity at a pH of about less than 2 in the stomach but lower activity at pH>5 [Piper D W, Fenton B H. pH stability and activity curves of pepsin with special reference to their clinical importance. Gut. 1965 October; 6 (5): 506-8. doi: 10.1136/gut.6.5.506. PMID: 4158734; PMCID: PMC1552331]. In the fasted stomach, the pH of the fasted gastric medium remains less than 2 (about 1.3)/Russell T L, Berardi R R, Barnett J L, Dermentzoglou L C, Jarvenpaa K M, Schmaltz S P, Dressman J B. Upper gastrointestinal pH in seventy-nine healthy, elderly, North American men and women. Pharm Res. 1993 February; 10 (2): 187-96. doi: 10.1023 a: 1018970323716. PMID: 8456064]. Therefore, the term “fasted pH of the gastric medium”, “pH of the fasted gastric medium”, or “pH of the gastric medium in the fasted stomach” refers to a pH of about 1.2 or less than about 2. Further, the fasted stomach contained about 35 mL of gastric fluid. Per US FDA guidelines, it is recommended that any pharmaceutical composition should be administered with 240 mL of drinking water. Upon drinking the 240 mL of water, the gastric fluid will increase to about 242 mL after 2 minutes in a fasted state [Quantification of Gastrointestinal Liquid Volumes and Distribution Following a 240 ml Dose of Water in the Fasted State, Mol. Pharmaceutics 2014, 11, 9, 3039-3047, https://doi.org/10.1021/mp500210c]. Further, in some instances, the dosage form, such as Rybelsus® (Semaglutide) tablet, is recommended to be administered with 120 mL of drinking water. Therefore, when the term “acidic medium”, “gastric fluid”, “gastric medium”, “fasted gastric medium”, or “stomach fluid” are used in this specification, it is in the meaning of 35 mL of any medium having a pH of about 1.2, with or without dilution with 120 mL or 240 mL of water, i.e., 35 ml of any medium having pH of about 1.2, or 35 mL of any medium having pH of about 1.2 diluted with the addition of either 120 mL or 240 mL of water. Thus, for in-vivo simulation of the gastric medium of the fasted stomach, the inventor considered 35 mL of any medium having a pH of about 1.2, or 35 mL of any medium having a pH of about 1.2 diluted with the addition of either 120 mL or 240 mL of water (e.g., 35 mL of 0.1N HCl+125 mL of water or 160 mL of any acidic medium having the pH of less than about 2).

As used in this specification, the term “gastrointestinal enzyme” refers to a gastric enzyme and one or more intestinal enzymes. As used in this specification, the term “upper part of the gastrointestinal tract/region” refers to the stomach, the duodenal region, or both. The term “upper part of the intestinal region/tract” refers to the duodenal region.

As used in this specification, the term “acid-neutralizing ingredient” or “acid-neutralizing agent” or “alkalizer” or “alkalizing agent” are used interchangeably to refer to any excipient or an ingredient that acts to increase the pH of the fasted gastric medium to any pH above about 2, for example, greater than about 3, preferably at least about 4, more preferably at least about 5, and most preferably at least about 6. Further, the type of acid-neutralizing agent does not limit the scope of the presently disclosed subject matters. For example, any substance or ingredient, including all of those which are not listed in this specification but that is capable of increasing the pH of the fasted gastric medium to greater than 2, greater than 2.5, greater than 4, greater than 4.5, greater than 5.5, or greater than 6 can be used as an acid-neutralizing agent. It also refers to a basic substance or agent that can convert an acidic environment to a less acidic, or more basic, environment. Typically, these agents, when present in a sufficient amount, are able to elevate the pH of the fasted gastric medium to above the threshold pH at which at least one of the drug and the alkaline salt of the oral absorption promoter is soluble, or elevate the pH of the fasted gastric medium to at least greater than about 2, preferably greater than 2.5 and more preferably greater than 5. The acid-neutralizing agent also includes surfactants such as sodium lauryl sulfate, and the like.

As used in this specification, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 15%, up to 10%, up to 5%, or up to 1% of a given value. Alternatively, “about” can mean within 3 or more than 3 standard deviations, per the practice in the art. Alternatively, particularly with respect to biological systems or processes, the term “about” can also mean within an order of magnitude, preferably within five-fold, and more preferably within two-fold, of a value.

As used in this specification, the terms “material”, “excipient”, “agent”, “additive”, “ingredient”, and “component” are interchangeable, and one of skill in the art is cognizant that these terms are open-ended terms.

The following is a detailed description of embodiments of the presently disclosed subject matter. The embodiments described herein may be used independently or in conjunction with any definition, claim, or any other embodiment mentioned herein. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims. Thus, the presently disclosed subject matter contemplates all possible combinations and permutations of the various independently described embodiments.

Each of the appended claims defines a separate invention, which for infringement purposes is recognized as including equivalents to the various elements or limitations specified in the claims. Depending on the context, all references below to the “invention” may, in some cases, refer to certain specific embodiments only. In other cases, it will be recognized that references to the “invention” will refer to subject matter recited in one or more, but not necessarily all, of the claims.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

SNAC contained in the RYBELSUS tablet [(semaglutide) tablets contained 7 mg or 14 mg of Semaglutide and SNAC] improved the absorption of Semaglutide when administered in the fasted state stomach; however, the variability in the Semaglutide exposure was found to be very high after a single dose, as some subjects had concentrations below the lower limit of quantification. The inventors of the published U.S. Pat. No. 11,759,501B2 found that the bioavailability of GLP-1 peptide, such as Semaglutide, from the different compositions comprising various granule designs of the SNAC can be predicted from the in vitro dissolution or solubilization behavior of the SNAC. The granules that offered an improved dissolution rate of the SNAC had a marked improvement in the bioavailability of GLP-1 peptide from the composition.

In certain embodiments, the presently disclosed pharmaceutical formulation, when administered in a fasted state, improves the oral absorption of a drug contained therein.

In certain embodiments, the presently disclosed pharmaceutical formulation, when administered in a fasted state, improves the solubility of a drug contained therein.

In certain embodiments, the presently disclosed pharmaceutical formulation, when administered in a fasted state, affords protection, at least in part, to a drug contained therein from fasted gastric degradation.

In certain embodiments, the presently disclosed pharmaceutical formulation, when administered in a fasted state, affords protection, at least in part, to a drug contained therein from fasted gastric degradation and further affords protection, at least in part, to the drug from proteolytic degradation.

In certain embodiments, the presently disclosed pharmaceutical formulation, when administered in a fasted state, elevates the pH of a fasted gastric medium (or for in-vitro measurement, elevates the pH of media such as 35 mL of 0.1N HCl, or a mixture of 35 mL of 0.1N HCl+125 mL of water, or 160 mL of acidic media having pH less than about 2) from less than about 2 to at least about 2.5, such as 3, 3.5, 4, 4.5, 5, 5.5, 6, 6.5, 7, or 7.5, but less than about 10, such as less than about 9, preferably less than about 8, within the first 60 minutes, preferably within 55 minutes, such as within 50, 45, 40, 35, 30, 25, 20, 15, 10, 8, 6, 5, 4, 3, 2, or 1 minute or less.

In certain embodiments, the presently disclosed pharmaceutical formulation, when administered in a fasted state, facilitates the enhanced dissolution of an alkaline salt of oral absorption promoter contained therein in a fasted gastric medium (or for in-vitro measurement, facilitates the enhanced dissolution of an alkaline salt of oral absorption promoter in the media such as 35 mL of 0.1N HCl, or mixture of 35 mL of 0.1N HCl+125 mL of water, or 160 mL of acidic media having pH less than about 2).

In preferred embodiment, the present invention relates to a pharmaceutical formulation that comprises a drug, an alkaline salt of an oral absorption promoter, an alkalizing agent, and optionally one or more pharmaceutically acceptable excipients. The terms “pharmaceutical formulation” and “pharmaceutical composition” can be used interchangeably and refer to an orally administrable pharmaceutical composition formulated for administration to a patient to treat a disease or condition, such as a composition formulated into a solid unitary dosage form (like a tablet or capsule), or a liquid formulation (powder for reconstitution, solution, suspension, emulsion, etc.), that comprises a drug and one or more ingredients (such as an alkaline salt of an oral absorption promoter, an alkalizing agent, an intestinal enzyme inhibitor, and one or more pharmaceutically acceptable excipients). The pharmaceutical formulation could be fabricated in any form that facilitates easy administration as a unit dose, which does not restrict the scope of the present invention; examples include mono- or multi-layer tablets, capsules (of any type, including capsule-in-capsule and hard or soft gelatin capsules), reconstituted powders, pellets, lozenges, films, powders, liquids, suspensions, emulsions and more.

The present inventors surprisingly found that the dissolution properties of an alkaline salt of an oral absorption promoter contained in the pharmaceutical formulation are greatly improved in an acidic medium (e.g., acidic medium having a pH of less than about 2, e.g., 35 mL of 0.1N HCl, or mixture of 35 mL of 0.1N HCl+125 mL of water, or 150 mL of acidic media having pH less than about 2), provided that at least one acid-neutralizing agent contained in the pharmaceutical formulation is released in the acidic medium prior to, concurrently with, or faster than the release of the alkaline salt of the oral absorption promoter from the pharmaceutical formulation. The present inventors surprisingly found that the dissolution properties of an alkaline salt of an oral absorption promoter contained in the pharmaceutical formulation are greatly improved in an acidic medium (e.g., acidic medium having a pH of less than about 2, e.g., 35 mL of 0.1N HCl, or mixture of 35 mL of 0.1N HCl+125 mL of water, or 150 mL of acidic media having pH less than about 2), provided that at least one acid-neutralizing agent contained in the pharmaceutical formulation is released in the acidic medium slower than, or after the release of the alkaline salt of the oral absorption promoter.

The present inventors surprisingly found that the dissolution properties of an alkaline salt of an oral absorption promoter contained in the pharmaceutical formulation are greatly improved in an acidic medium having pH of less than 2 (but pH of greater than about 1) (e.g., acidic medium having a pH of less than about 2, e.g., 35 mL of 0.1N HCl, or mixture of 35 mL of 0.1N HCl+125 mL of water, or 150 mL of acidic media having pH less than about 2), provided that the said pharmaceutical formulation is comprised of at least one acid neutralizer in the amount that is at least sufficient to elevate the pH of the acidic medium to at least 2 or greater [preferably elevate the pH to at least about 2.5, such as at least about 3, at least about 4, at least about 5, and more preferably at least about 5.5 or higher, but less than about 8], and releases the said at least one acid neutralizer in the acidic medium prior to, concurrently with, or faster than the release of the alkaline salt of the oral absorption promoter. The present inventors also surprisingly found that the dissolution property of an alkaline salt of oral absorption promoter contained in the pharmaceutical formulation is markedly improved in an acidic medium having pH of less than 2 (but pH of greater than about 1) (e.g., acidic medium having a pH of less than about 2, e.g., 35 mL of 0.1N HCl or mixture of 35 mL of 0.1N HCl+125 mL of Water or 150 mL of acidic media having pH less than about 2), provided that the said pharmaceutical formulation is comprised of at least one acid neutralizer in the amount that is at least sufficient to elevate the pH of the acidic medium to at least 2 or greater [preferably elevate the pH to at least about 2.5, such as at least about 3, at least about 4, at least about 5, and more preferably at least about 5.5 or higher but less than about 8], and releases the said at least one acid neutralizer in the acidic medium slower than or after the release of the alkaline salt of the oral absorption promoter.

The inventors of the published U.S. Pat. No. 11,759,501B2 found that the bioavailability of drug, such as GLP-1 peptide, more specifically Semaglutide, from the different pharmaceutical compositions comprising various granule designs of the SNAC (alkaline salt of oral absorption promoter) can be predicted from the in vitro dissolution or solubilization behavior of the SNAC. The granules that offered an improved dissolution rate of the SNAC had a marked improvement in the bioavailability of GLP-1 peptide from the composition.

The present inventor surprisingly found that the dissolution behavior of the alkaline salt of oral absorption promoter incorporated in the pharmaceutical formulation, such as SNAC, could be improved in an acidic medium having the pH of less than about 2 (but greater than about 1), such as in the fasted gastric medium when administered in the fasted stomach, which can subsequently greatly improve the oral bioavailability of the drug contained in the pharmaceutical formulation, provided that the said pharmaceutical formulation contained at least one acid-neutralizing agent in the amount that is at least sufficient to elevate the pH of the acidic medium to at least 2 or greater [preferably elevate the pH to at least about 2.5, such as at least about 3, at least about 4, at least about 5, and more preferably at least about 5.5 or higher but less than about 8] and it releases the acid neutralizing agent in the acidic medium prior to, concurrently with, faster than, slower than or after the release of the alkaline salt of the oral absorption promoter from the pharmaceutical formulation.

The present inventors found that improved dissolution behavior of the alkaline salt of oral absorption promoter, such as SNAC, in the acidic medium having the pH of less than about 2 (but >1), such as in the fasted gastric medium when administered in the fasted stomach, can significantly improve the oral bioavailability of the drug contained in the pharmaceutical formulation when it is administered in the fasted state stomach.

In preferred embodiment, the present invention relates to a pharmaceutical formulation that comprises a drug, an alkaline salt of an oral absorption promoter, an acid-neutralizing agent (alkalizing agent), and optionally one or more pharmaceutically acceptable excipients, wherein the pharmaceutical formulation releases the acid neutralizing agent prior to, concurrently with, faster than, slower than or after the release of the alkaline salt of the oral absorption promoter.

In certain embodiment, the present invention relates to a pharmaceutical formulation that comprises a drug, 5-95% w/w of an alkaline salt of an oral absorption promoter, an acid-neutralizing agent in the amount that is at least sufficient to elevate the pH of mixture of 35 mL of 0.1N HCl and 125 ml of water from less than about 2 to at least about 2.5 (such as 3, 3.5, 4, 4.5, 5, 5.5, 6, 6.5, 7, or 7.5, but less than about 10, such as less than about 9), and optionally one or more pharmaceutically acceptable excipients, wherein the said pharmaceutical formulation when subjected to in-vitro dissolution in the mixture of 35 mL of 0.1N HCl and 125 ml of water (dissolution medium), using USP type II/I apparatus (paddle/basket, respectively), or equivalent dissolution apparatus in design (such as using smaller size paddle/basket or using smaller size dissolution vessel), at 50/75/100 RPM and 37° C.±0.5° C. temperature, it releases the acid neutralizing agent prior to, concurrently with, faster than, slower than or after the release of the alkaline salt of the oral absorption promoter, which subsequently greatly improves the dissolution behavior of the alkaline salt of the oral absorption promoter in the dissolution medium.

In the present invention, the term “acidic medium” refers to 35 mL of 0.1N HCl or 160 mL of any aqueous media having a pH of less than about 2 (the pH of less than about 2 means any of the pH between 1.5, 1.6, 1.7, 1.8, 1.9 or less than about 2, preferably pH about 1.7), e.g., 35 mL of 0.1N HCl or a mixture containing 35 mL of 0.1N HCl+125 mL of water, which mimics the fasted state stomach condition, such as pH and volume of gastric fluid in the fasted stomach state or the condition of the fasted state stomach after administration of 125 ml of water, respectively. During the fasted state, the stomach contains about 35 mL of resting gastric medium [Quantification of Gastrointestinal Liquid Volumes and Distribution Following a 240 ml Dose of Water in the Fasted State, Mol. Pharmaceutics 2014, 11, 9, 3039 3047, https://doi.org/10.1021/mp500210c] having the pH of less than about 2 (e.g., about 1.3, from 1.1 to 1.6)/Russell T L, Berardi R R, Barnett J L, Dermentzoglou L C, Jarvenpaa K M, Schmaltz S P, Dressman J B. Upper gastrointestinal pH in seventy-nine healthy, elderly, North American men and women. Pharm Res. 1993 February; 10 (2): 187-96. doi: 10.1023/a: 1018970323716].

In the present invention, the term “acidic medium” also refers to 160 mL of any aqueous media having a pH of about 1.7.

In certain embodiment, the present invention relates to a pharmaceutical formulation that facilitates the enhanced dissolution of an alkaline salt of an oral absorption promoter contained therein, wherein the said pharmaceutical formulation comprises (1) a drug in the amount of about 0.01-85%, such as 0.1-75%, 0.5-70%, 0.75-60%, 1-55%, 1.5-50% (w/w) of the total amount of the pharmaceutical formulation, (2) an alkaline salt of an oral absorption promoter in the amount of about 1-99%, such as 5-98%, 10-96%, 15-95%, 30-90%, 35-85% (w/w) of the total amount of the pharmaceutical formulation, (3) an acid neutralizer in an amount of about 1-75%, 2-65%, 2.5-50%, 3-40%, 3.5-35% (w/w) of the total amount of the pharmaceutical formulation or in an amount that is at least sufficient to elevate the pH of mixture of 35 mL of 0.1N HCl and 125 ml of water from less than about 2 to at least about 2.5 (such as 3, 3.5, 4, 4.5, 5, 5.5, 6, 6.5, 7, or 7.5, but less than about 10, such as less than about 9, preferably less than about 8), and (4) optionally one or more pharmaceutically acceptable excipients, wherein the said pharmaceutical formulation when subjected to in-vitro dissolution in the mixture of 35 mL of 0.1N HCl and 125 ml of water (dissolution medium), using USP type II/I apparatus (paddle/basket, respectively) or equivalent dissolution apparatus in design (such as using smaller size paddle/basket or using smaller size dissolution vessel), at 50/75/100 RPM and 37° C.±0.5° C. temperature, it releases the acid neutralizing agent prior to, concurrently with, faster than, slower than or after the release of the alkaline salt of the oral absorption promoter, which subsequently greatly improves the dissolution behavior of the alkaline salt of the oral absorption promoter in the dissolution medium.

The terms “acid neutralizer” or “acid neutralizing agent” are used interchangeably. In the present invention, “in-vitro release test” or the term “release” when used with reference to the acid neutralizer and optionally compared to release of the alkaline salt of an oral absorption promoter, is meant to be determined as solubility (% assay) of alkaline salt of an oral absorption promoter within 45 minutes (such as within 45, 30, 25, 20, 15, 10, 5, 2.5, or 1 minutes), as a function of sequence of addition of the acid neutralizer, such as addition of the acid neutralizer concurrently with, prior to, faster than, slower than, or after the addition of alkaline salt of an oral absorption promoter in 35 mL of 0.1N HCl or mixture of 35 mL of 0.1N HCl and 125 ml of water, with gentle shaking of the resultant mixture in the container or under continuous stirring of the resultant mixture. “In-vitro release test” or the term “release” when used with reference to the acid neutralizer and optionally compared to release of the alkaline salt of an oral absorption promoter, is also mean to be determined as dissolution (% dissolved content) of alkaline salt of an oral absorption promoter within 45 minutes (such as within 45, 30, 25, 20, 15, 10, 5, 2.5, or 1 minutes), as a function of sequence of addition of the acid neutralizer, such as addition of the acid neutralizer concurrently with, prior to, faster than, slower than, or after the addition of alkaline salt of an oral absorption promoter in the mixture of 35 mL of 0.1N HCl and 125 ml of water (dissolution medium), using USP type II/I apparatus (paddle/basket, respectively), or equivalent dissolution apparatus in design (such as using smaller size paddle/basket or using smaller size dissolution vessel), at 50/75/100 RPM and 37° C.±0.5° C. temperature.

In certain embodiment, the present invention relates to a pharmaceutical formulation that facilitates the enhanced dissolution of an alkaline salt of an oral absorption promoter contained therein, wherein the said pharmaceutical formulation comprises (1) a drug in the amount of about 0.01-85%, such as 0.1-75%, 0.5-70%, 0.75-60%, 1-55%, 1.5-50% (w/w) of the total amount of the pharmaceutical formulation, (2) an alkaline salt of an oral absorption promoter in the amount of about 1-99%, such as 5-98%, 10-96%, 15-95%, 30-90%, 35-85% (w/w) of the total amount of the pharmaceutical formulation, (3) an acid neutralizer in an amount of about 1-75%, 2-65%, 2.5-50%, 3-40%, 3.5-35% (w/w) of the total amount of the pharmaceutical formulation or in an amount that is at least sufficient to elevate the pH of 35 mL or 160 mL of acidic medium from less than about 2 to at least about 2.5 (such as 3, 3.5, 4, 4.5, 5, 5.5, 6, 6.5, 7, or 7.5, but less than about 10, such as less than about 9, preferably less than about 8), and (4) optionally one or more pharmaceutically acceptable excipients, wherein the said pharmaceutical formulation when subjected to in-vitro release test in the 35 mL or 160 mL of acidic medium, releases the acid neutralizing agent prior to, concurrently with, faster than, slower than or after the release of the alkaline salt of the oral absorption promoter, which subsequently greatly improves the dissolution behavior of the alkaline salt of an oral absorption promoter in the 35 mL or 160 mL of acidic medium, thereby facilitating marked improvement in the release or dissolution of the alkaline salt of an oral absorption promoter in the 35 mL or 160 mL of the acidic medium.

The present inventors surprising found that the sequence of the release of the acid neutralizer in compared to the release of the alkaline salt of an oral absorption promoter (such as release of acid neutralizer prior to release of alkaline salt of an oral absorption promoter, release of acid neutralizer concurrently with release of alkaline salt of an oral absorption promoter, release of acid neutralizer after release of alkaline salt of an oral absorption promoter, release of acid neutralizer faster than release of alkaline salt of an oral absorption promoter, release of acid neutralizer slower than release of alkaline salt of an oral absorption promoter, etc.) from the pharmaceutical formulation had a marked effect on the release or dissolution behavior of the alkaline salt of an oral absorption promoter in the 35 mL or 160 mL of the acidic medium having a pH of less than about 2 (such as in fasted state stomach condition). For example, the acid neutralizer that completely released prior to the start of release of the alkaline salt of an oral absorption promoter provided 72.94% release or dissolution of the alkaline salt of an oral absorption promoter at 2 minutes in 160 mL of the acidic medium, the acid neutralizer that released concurrently with the release of the alkaline salt of an oral absorption promoter provided 10.287% release or dissolution of the alkaline salt of an oral absorption promoter at 2 minutes in 160 mL of the acidic medium, and the acid neutralizer that released after the complete release of alkaline salt of an oral absorption promoter provided only 3.65% release or dissolution of the alkaline salt of an oral absorption promoter at 2 minutes in 35 mL or 160 mL of the acidic medium, however, in all cases, the percentage release or dissolution of the alkaline salt of the oral absorption promoter at 2 minutes in 35 mL or 160 mL of the acidic medium is significantly enhanced compared to the absence of the acid neutralizer at 2 minutes, which was 0.013% (refer to the examples of the present application).

The breadth of the current invention is not restricted by how a release pattern of the acid neutralizer is incorporated into the pharmaceutical formulation in comparison to alkaline salts of an oral absorption promoter until it facilitates marked improvement in the release or dissolution of the alkaline salt of oral absorption promoter in the 35 mL or 160 mL of the acidic medium.

The acid neutralizer can be incorporated into the pharmaceutical composition in a number of ways. For example, it can be incorporated as a coating around the core (mono or multilayer tablet or pellets) of the drug and/or alkaline salts of an oral absorption promoter, or as a separate layer (either in immediate release or modified release form) in multi-layer tablets or pellets (such as mini-tablets), in solution or suspension form [which can also be fillable (along with drug and alkaline salts of an oral absorption promoter) in soft gelatin or hard gelatin capsules], in powder form, filled in capsules, in form of mouth dissolving film for oral administration, etc. Acid neutralizer can be made as individual granules and then combined with granules of drug and/or alkaline salts of an oral absorption promoter. In some formulations, the acid neutralizer might be included as a separate region within the pharmaceutical formulation to inhibit or reduce the degradation of any drug or alkaline salt of an oral absorption promoter.

In some embodiments the term “granule” refers to as such individual particle or two or more particles are gathered into one larger particle. The term granules refer to more than one individual particles or large particles as described previously.

In some embodiments, the invention relates to a pharmaceutical formulation according to any one of the preceding embodiments, wherein the acid neutralizer elevates the pH of 35 mL or 160 mL of acidic medium from less than about 2 to at least about 2.5, such as 3, 3.5, 4, 4.5, 5, 5.5, 6, 6.5, 7, or 7.5, but less than about 8, within the first 60 minutes, preferably within 55 minutes, such as within 50, 45, 40, 35, 30, 25, 20, 15, 10, 8, 6, 5, 4, 3, 2, or 1 minute or less, as determined by a standard pH meter (such as at ambient temperature or preferably at 25° C.).

In certain embodiments, the presently disclosed subject matters relate to a pharmaceutical formulation comprising a drug, an acid neutralizer in an amount that is at least sufficient to elevate the pH of 35 mL or 160 mL of acidic medium from less than about 2 to at least about 5 but less than about 8, and optionally one or more pharmaceutically acceptable excipients, wherein the said pharmaceutical formulation when administered in the fasted state of the stomach, the acid neutralizer elevates the pH of fasted gastric medium from less than about 2 to lesser acidic pH of greater than about 5, thereby it (a) improves the solubility of the drug in the lesser acidic fasted gastric medium, and/or, (b) affords protection, at least in part, to the drug from fasted gastric degradation.

In certain embodiments, the presently disclosed subject matters relate to a pharmaceutical formulation comprising a drug, an acid neutralizer in an amount that is at least sufficient to elevate the pH of 35 mL or 160 mL of acidic medium from less than about 2 to at least about 5 but less than about 8, an alkaline salt of an oral absorption promoter, and optionally one or more pharmaceutically acceptable excipients, wherein the said pharmaceutical formulation when administered in the fasted state of the stomach, the acid neutralizer elevates the pH of fasted gastric medium from less than about 2 to lesser acidic pH of greater than about 5, thereby it (a) improves the solubility of the drug in the lesser acidic fasted gastric medium, (b) affords protection, at least in part, to the drug from fasted gastric degradation, (c) facilitates the enhanced dissolution of the alkaline salt of an oral absorption promoter in the lesser acidic fasted gastric medium (or media such as e.g., in 35 mL of 0.1N HCl, or a mixture of 35 mL of 0.1N HCl and 125 ml of water, or 160 mL of acidic media having pH less than about 2), or (d) any combination thereof.

In some embodiments, the pharmaceutical formulation of any of the previously disclosed embodiments further comprises a pharmaceutically acceptable amount of at least one intestinal enzyme inhibitor to further afford the protection, at least in part, to the drug from proteolytic degradation.

In certain embodiments, the intestinal enzyme inhibitors include at least one metal in the form of any or a combination of a salt thereof and a complex thereof, or a combination of (1) at least one metal in the form of any or a combination of a salt thereof and a complex thereof; and (2) at least one reducing agent.

In certain embodiments, the pharmaceutical formulation is a non-enteric formulation, an enteric formulation, or a partially non-enteric formulation. The partially non-enteric formulation begins a release of the drug in the non-enteric region, releases a substantial amount of acid neutralizer in the non-enteric region, and releases a substantial amount of the intestinal enzyme inhibitor in the enteric region.

In certain embodiments, the pharmaceutical formulation releases a substantial amount of the intestinal enzyme inhibitor in a gastric region (non-enteric region). In certain embodiments, the pharmaceutical formulation releases a substantial amount of the intestinal enzyme inhibitor in a non-gastric region (enteric region), i.e., any part of an intestinal region, preferably in the upper part of the intestinal region.

In certain embodiments, the pharmaceutical formulation contains more than one region or plurality of regions, wherein at least one region (e.g., in form of layer, coat, bead, pellet, particle, etc.) contains the intestinal enzyme inhibitor.

In certain embodiments, the intestinal enzyme inhibitor included in the pharmaceutical formulation is one or more coated cores, wherein each coated core is comprised of: a core comprised of at least one metal in the form of any or a combination of a salt thereof and a complex thereof, or a combination of (1) at least one metal in the form of any or a combination of a salt thereof and a complex thereof; and (2) at least one reducing agent, and an enteric coat surrounding the core, wherein the enteric coat is comprised of at least one enteric coating material.

Overall, protecting the drug from fasted gastric degradation, facilitating the enhanced dissolution of the alkaline salt of an oral absorption promoter when administered in fasted state stomach (with or without 125 ml of water), and further protecting the drug from proteolytic degradation in the intestinal region can overall enhance the absorption (and thus bioavailability) of the drug contained in the pharmaceutical formulation to a greater extent. Further, per the invention, desired therapeutic efficacy can be achieved by using a lower amount of the alkaline salt of an oral absorption promoter if the pharmaceutical formulation comprises the acid neutralizer in an amount that is at least sufficient to elevate the pH of 35 mL or 160 mL of acidic medium from less than about 2 to at least about 2.5, such as at least about 3.5, at least about 4.5, at least about 5.5, but less than about 8. The enhanced oral absorption of the drug can be determined by a comparative in-vivo bioavailability study.

In the present invention, % w/w (wt %) of any of the ingredients (including drug) in the total weight of the pharmaceutical composition can be adjusted without any limitation as required and as known to or appreciated by a person skilled in the art, without departing from the scope and spirit of the present invention.

In some embodiments, the pharmaceutical formulation comprises the drug in the amount of about 0.01-85%, such as 0.1-75%, 0.5-70%, 0.75-60%, 1-55%, 1.5-50% (w/w) of the total amount of the pharmaceutical formulation. In some embodiments, the pharmaceutical formulation comprises the drug in an amount of more than about 0.01%, such as >0.1%, >1%, >2%, >5%, >10%, >15%, >20%, >25%, >35%, but less than about of 85%, such as <75%, <65%, <55%, <50%, <40% (w/w) of the total amount of the pharmaceutical formulation.

In certain embodiments, the drug exhibits a molecular weight of equal to or less than about 60 kDa. In certain embodiments, the drug exhibits a molecular weight of equal to or less than about 40 kDa. In certain embodiments, the drug exhibits a molecular weight of equal to or less than about 20 kDa. In certain embodiments, the drug exhibits a molecular weight of equal to or less than about 10 kDa. In certain embodiments, the drug exhibits a molecular weight ranging from about equal to or greater than about 150 Da to about equal to or less than about 60 kDa, such as less than 40 kDa, 20 kDa, 10 kDa, 5 kDa. However, the drug with any range of molecular weight, as known to or appreciated by a person skilled in the art, can be utilized to serve its intended purpose, as laid in the present disclosure, without departing from the scope and spirit of the present invention. In certain embodiments, the molecular weight of the drug can be determined by any method such as mass spectrometry (electrospray ionization mass spectrometry (ESI-MS) or matrix-assisted laser desorption/ionization mass spectrometry (MALDI-MS)), gel electrophoresis (polyacrylamide gel electrophoresis using sodium dodecyl sulfate (SDS-PAGE)), hydrodynamic methods (gel filtration chromatography or gradient sedimentation), or static light scattering (e.g., multi-angle light scattering (MALS), known to or appreciated by a person skilled in the art, to serve its intended purpose, as laid in the present disclosure, without departing from the scope and spirit of the present invention.

In certain embodiments, the drug includes, but are not limited to, analgesics, anti-inflammatory agents, anti-helminthics, anti-arrhythmic agents, anti-asthma agents, anti-bacterial agents, anti-viral agents, anti-coagulants, anti-dementia agents, anti-depressants, anti-diabetics, anti-epileptics, anti-fungal agents, anti-gout agents, anti-hypertensive agents, anti-malarial agents, anti-migraine agents, anti-muscarinic agents, anti-neoplastic agents, immunosuppressants, antiprotozoal agents, anti-thyroid agents, antitussives, anxiolytics, sedatives, hypnotics, neuroleptics, neuroprotective agents, β-blockers, cardiac inotropic agents, cell adhesion inhibitors, corticosteroids, cytokine receptor activity modulators, diuretics, anti-Parkinson's agents, gastro-intestinal agents, histamine H-receptor antagonists, keratolyses, lipid regulating agents, muscle relaxants, nitrates and other anti-anginal agents, non-steroid anti-asthma agents, nutritional agents, opioid analgesics, sex hormones, stimulants and anti-erectile dysfunction agents; and salts, esters, and mixtures thereof.

In certain embodiments, the drug includes, but is not limited to, peptides, glucagon-like peptide 1 (GLP 1) agonist, hormones, polysaccharides, carbohydrates, lipids, or any derivative or analogues thereof.

In certain embodiments, the drug includes, but is not limited to, human growth hormones; bovine growth hormones; growth releasing hormones; interferons; interleukin-1; insulin; heparin and particularly low molecular weight heparin; calcitonin; peptide and particularly small peptide; glucagon-like peptide 1 (GLP 1) agonist; erythropoietin; atrial natriuretic factor; antigens; monoclonal antibodies; somatostatin; adrenocorticotropin, gonadotropin releasing hormone; oxytocin; vasopressin; cromolyn sodium (sodium or disodium chromoglycate); vancomycin; desferrioxamine (DFO); parathyroid hormone anti-microbials, including, but not limited to anti-fungal agents; or any derivative or analogues thereof.

In certain embodiments, the drug includes one or more of the drugs mentioned in WO1993/019175, WO1996/029342, WO1998/008871, WO1999/043707, WO1999/043706, WO1999/043341, WO1999/043708, WO2005/027978, WO2005/058954, WO2005/058958, WO2006/005667, WO2006/037810, WO2006/037811, WO2006/097537, WO2006/097538, WO2008/023050, WO2009/030738, WO2009/030771, WO2009/030774, U.S. Pat. No. 10,960,052B2, U.S. Pat. No. 11,389,474B2 and EP3006045B1, the entire published patent applications or granted patents herein incorporated by reference.

In certain embodiments, the drug is a peptide, and the peptide, according to the present invention, is selected from one or more of the peptides mentioned in US20200121796A1 and US20170304195A1, the entire published patent application herein incorporated by reference. In certain embodiments, the drug is at least one peptide with a molecular weight ranging from about equal to or greater than about 150 Da to about equal to or less than about 60 kDa. In certain embodiments, the peptide is selected from any or a combination of insulin (preferably human insulin), an insulin analog such as, but not limited to, a long acting basal insulin analog, a protease stabilized long acting basal insulin analog, insulin lispro, insulin PEGlispro, the insulin derivative like A14E, B25H, B29K(N(eps)octadecanedioyl-gGlu-OEG-OEG), desB30 human insulin, B29K(N(ε)hexadecanedioyl-γ-L-Glu) A14E B25H desB30 human insulin, B29K(N(ε)octadecanedioyl-γ-L-Glu-OEG-OEG) desB30 human insulin, B29K(N(ε)octadecanedioyl-γ-L-Glu) A14E B25H desB30 human insulin, B29K(N(ε)eicosanedioyl-γ-L-Glu) A14E B25H desB30 human insulin, B29K(N(ε)octadecanedioyl-γ-L-Glu-OEG-OEG) A14E B25H desB30 human insulin, B29K(N(ε)eicosanedioyl-γ-L-Glu-OEG-OEG) A14E B25H desB30 human insulin, B29K(N(ε)eicosanedioyl-γ-L-Glu-OEG-OEG) A14E B16H B25H desB30 human insulin, B29K(N(ε)hexadecanedioyl-γ-L-Glu) A14E B16H B25H desB30 human insulin, B29K(N(ε)eicosanedioyl-γ-L-Glu-OEG-OEG) A14E B16H B25H desB30 human insulin, B29K(N(ε)octadecanedioyl) A14E B25H desB30 human insulin, insulin aspart, insulin glulisine, insulin glargine, insulin detemir, NPH insulin, insulin degludec, and the insulin analogs/derivatives described in US application number US20140056953A1, GLP-1, a GLP-1 analog, acylated GLP-described in US application number US20140056953A1, GLP-1, a GLP-1 analog, acylated GLP-1 analog, a diacylated GLP-1 analog, semaglutide, liraglutide, exenatide, lixizenatide, orforglipron, a dual agonist of the GLP-1 receptor and the glucagon receptor, amylin, an amylin analog, pramlintide, a somatostatin analog (octreotide, lanreotide, or pasireotide), goserelin (goserelin acetate), buserelin, leptin, a leptin analog (metreleptin), peptide YY (PYY), a PYY analog, glatiramer (glatiramer acetate), leuprolide, teriparatide, abaloparatide, tetracosactide, corticorelin, etelcalcetide, elcatonin, desmopressin, human growth hormone (hGH), a human growth hormone analog, a glycopeptide antibiotic (a glycosylated cyclic or polycyclic nonribosomal peptide such as vancomycin, teicoplanin, telavancin, bleomycin, ramoplanin, or decaplanin), bortezomib, cosyntropin, chorionic gonadotropin, menotropin, sermorelin, luteinizing-hormone-releasing hormone (LHRH, also referred as gonadotropin-releasing hormone), somatropin, calcitonin (calcitonin-salmon), pentagastrin, oxytocin, neseritide, anakinra, enfuvirtide, pegvisomant, dornase alfa, lepirudin, anidulafungin, eptifibatide, interferon alfacon-1, interferon alpha-2a, interferon alpha-2b, interferon beta-1a, interferon beta-1b, interferon gamma-1b, peginterferon alfa-2a (pegylated interferon alfa-2a), peginterferon alfa-2b (pegylated interferon alfa-2b), peginterferon beta-1a (pegylated interferon beta-1a), fibrinolysin, vasopressin, aldesleukin, epoetin alfa, darbepoetin alfa, epoetin beta, epoetin delta, epoetin omega, epoetin zeta, filgrastim, interleukin-11, cyclosporine, glucagon, urokinase, viomycin, thyrotropin-releasing hormone (TRH), leucine-enkephalin, methionine-enkephalin, substance P (CAS no. 33507-63-0), adrenocorticotropic hormone (ACTH), parathyroid hormone (PTH), and pharmaceutically acceptable salts thereof. However, any other peptide molecule with a molecular weight ranging from about equal to or greater than about 150 Da to about equal to or less than about 60 kDa, as known to or appreciated by a person skilled in the art, can be utilized to serve its intended purpose, as laid in the present disclosure, without departing from the scope and spirit of the present invention.

In certain embodiments, the drug is selected from any or a combination of endogenous peptides such as insulin or glucagon and the likes. In certain preferred embodiments, a human isoform of the corresponding peptide that is recombinantly expressed or chemically synthesized is used. However, any other human isoform peptide, as known to or appreciated by a person skilled in the art, can be utilized to serve its intended purpose, as laid in the present disclosure, without departing from the scope and spirit of the present invention.

In certain embodiments, the drug is a peptide, wherein the peptide is an insulin analog. In certain embodiments, the insulin analog is selected from any or a combination of insulin Detemir, insulin glargine, insulin degludec, and other insulin analogs derived from human, porcine, and fish. However, any other insulin analog/derivative, as known to or appreciated by a person skilled in the art, can be utilized to serve its intended purpose, as laid in the present disclosure, without departing from the scope and spirit of the present disclosure. In certain embodiments, a mixture of two or more peptides can be used. In an embodiment, a mixture of human insulin and a GLP-1 agonist (e.g., liraglutide, semaglutide, exenatideor, lixizenatide) is used. However, a mixture of any two or more peptides (including the peptides discussed above), as known to or appreciated by a person skilled in the art, can be utilized to serve its intended purpose, as laid in the present disclosure, without departing from the scope and spirit of the present invention.

In some embodiments the drug is selected from one or more of the GLP-1 peptides mentioned in WO93/19175, WO96/29342, WO98/08871, WO99/43707, WO99/43706, WO99/43341, WO99/43708, WO2005/027978, WO2005/058954, WO2005/058958, WO2006/005667, WO2006/037810, WO2006/037811, WO2006/097537, WO2006/097538, WO2008/023050, WO2009/030738, WO2009/030771 and WO2009/030774. (71) In some embodiments the GLP-1 peptide is selected from the group consisting of N-epsilon 37{2-[2-(2-{2-[2-((R)-3-carboxy-3-{[1-(19-carboxynonadecanoyl)piperidine-4-carbonyl]amino}propionylamino)ethoxy]ethoxy}acetylamino)ethoxy]ethoxy}acetyl [desaminoHis7,Glu22,Arg26,Arg34,Lys37]GLP-1(7-37)amide; N-epsilon26{2-[2-(2-{2-[2-((R)-3-carboxy-3-{[1-(19-carboxynonadecanoyl) piperidine-4-carbonyl]amino}propionylamino)ethoxy]ethoxy}acetylamino)ethoxy]ethoxy}acetyl[desaminoHis7, Arg34]GLP-1-(7-37); N-epsilon37{2-[2-(2-{2-[2-((S)-3-carboxy-3-{[1-(19-carboxy-nonadecanoyl) piperidine-4-carbonyl]amino}propionylamino)ethoxy]ethoxy}acetylamino)ethoxy]ethoxy}acetyl[Aib8,Glu22, Arg26,Arg34,Lys37]GLP-1-(7-37)amide; N-epsilon37-[2-(2-[2-(2-[2-(2-((R)-3-[1-(17-carboxyheptadecanoyl)piperidin-4-ylcarbonylamino]3-carboxypropionylamino)ethoxy)ethoxy]acetylamino)ethoxy]ethoxy)acetyl][,DesaminoHis7, Glu22 Arg26, Arg 34, Phe(m-CF3)28]GLP-1-(7-37)amide; N-epsilon26-[(S)-4-carboxy-4-({trans-4-[(19-carboxynonadecanoylamino)methyl]cyclohexanecarbonyl}amino)butyryl][Aib8,Arg34]GLP-1-(7-37); N-epsilon26-{4-[(S)-4-carboxy-4-({trans-4-[(19-carboxynonadecanoylamino)methyl]cyclohexanecarbonyl}amino)butyrylamino]butyryl}[Aib8,Arg34]GLP-1-(7-37); N-epsilon26-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetyl][Aib8,Arg34]GLP-1-(7-37); N-epsilon26-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetylamino]ethoxy}ethoxy) acetyl][Aib8,Arg34]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl][Aib8,Glu22,Arg26, Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl][DesaminoHis7,Glu22, Arg26,Arg34,Lys37]GLP-1-(7-37)amide; N-epsilon37-[2-(2-{2-[2-(2-{2-[(S)-4-carboxy-4-({4-[(trans-19-carboxy-nonadecanoylamino)methyl]cyclohexanecarbonyl}amino) butyrylamino]ethoxy}ethoxy)acetylamino]ethoxy}ethoxy)acetyl][DesaminoHis7,Arg26,Arg34,Lys 37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl][DesaminoHis7,Glu22,Arg26,Arg 34,Lys37]GLP-1-(7-37); N-epsilon26-[2-(2-{2-[2-(2-{2-[(S)-4-carboxy-4-({4-[(19-carboxy-nonadecanoylamino)methyl]cyclohexanecarbonyl}amino)butyrylamino]ethoxy}ethoxy) acetylamino]ethoxy}ethoxy)acetyl[Aib8, Lys 26]GLP-1 (7-37)amide; N-epsilon26-[2-(2-[2-(2-[2-(2-((S)-2-[trans-4-((9-carboxynonadecanoylamino]methyl)cyclohexylcarbonylamino]-4-carboxybutanoylamino)ethoxy)ethoxy]acetylamino) ethoxy]ethoxy)acetyl][Aib8, Lys26]GLP-1 (7-37)amide; N-epsilon37-[2-(2-{2-[2-(2-{2-[(S)-4-carboxy-4-({trans-4-[(19-carboxy-nonadecanoylamino)methyl]cyclohexane-carbonyl}amino)butyrylamino]ethoxy}ethoxy)acetylamino]ethoxy}ethoxy)acetyl][DesaminoHis7,Arg26,Arg34,Lys37]GLP-1-(7-37); N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetylamino]ethoxy}ethoxy)acetyl][DesaminoHis7,Glu22, Arg26,Glu30,Arg34,Lys37]GLP-1-(7-37); N-epsilon26-[2-(2-{2-[(S)-4-carboxy-4-((S)-4-carboxy-4-{4-[4-(16-(1H-tetrazol-5-yl)-hexadecanoylsulfamoyl)butyrylamino]-butyrylamino}butyrylamino) butyrylamino]ethoxy}ethoxy)acetyl][Aib8,Arg34]GLP-1-(7-37); N-epsilon26-[2-(2-{2-[(S)-4-carboxy-4-((S)-4-carboxy-4-{12-[4-(16-(1H-tetrazol-5-yl)hexadecanoyl-sulfamoyl)butyrylamino]dodecanoylamino}butyrylamino) butyrylamino]ethoxy}ethoxy)acetyl][Aib8,Arg34]GLP-1-(7-37); N-epsilon26-[2-(2-{2-[(S)-4-carboxy-4-((S)-4-carboxy-4-{6-[4-(16-(1H-tetrazol-5-yl)hexadecanoyl-sulfamoyl)butyrylamino]hexanoylamino}butyrylamino)butyrylamino]ethoxy}ethoxy) acetyl][Aib8,Arg34]GLP-1-(7-37); N-epsilon26-[2-(2-{2-[(S)-4-carboxy-4-((S)-4-carboxy-4-{4-[4-(16-(1H-tetrazol-5-yl)hexadecanoylsulfamoyl)butyrylamino]butyrylamino}butyrylamino)butyrylamino]ethoxy}ethoxy)acetyl][Aib8,Arg34]GLP-1-(7-34); N-epsilon26-[2-(2-{2[(S)-4-carboxy-4-((S)-4-carboxy-4-{12-[4-(16-(1H-tetrazol-5-yl)hexadecanoylsulfamoyl)butyrylamino]-dodecanoylamino}butyrylamino) butyrylamino]ethoxy}ethoxy)acetyl][Aib8,Arg34]GLP-1-(7-34); N-epsilon26-[2-(2-{2-[(S)-4-carboxy-4-((S)-4-carboxy-4-{6-[4-(16-(1H-tetrazol-5-yl)hexadecanoylsulfamoyl) butyrylamino]hexanoylamino}butyrylamino) butyrylamino]ethoxy}ethoxy)acetyl][Aib8,Arg34]GLP-1-(7-34); N-epsilon26-[2-(2-{2-[(S)-4-carboxy-4-((S)-4-carboxy-4-{12-[4-(16-(1H-tetrazol-5-yl)hexadecanoyl-sulfamoyl)butyrylamino]dodecanoylamino}butyrylamino)butyrylamino]ethoxy}ethoxy)acetyl][Aib8,Arg34]GLP-1-(7-35); N-epsilon26-[2-(2-{2-[(S)-4-carboxy-4-((S)-4-carboxy-4-{6-[4-(16-(1H-tetrazol-5-yl)hexadecanoylsulfamoyl)butyrylamino]hexanoylamino}butyrylamino)butyrylamino]ethoxy}ethoxy)acetyl][Aib8,Arg34]GLP-1-(7-35); N-epsilon26-[2-(2-{2-[(S)-4-carboxy-4-((S)-4-carboxy-4-{-6-[4-(16-(1H-tetrazol-5-yl)hexadecanoylsulfamoyl)butyrylamino]hexanoylamino}butyrylamino)butyrylamino]ethoxy}ethoxy)acetyl][Aib8,Arg34]GLP-1-(7-36)amide; N-epsilon26-[2-(2-{2-[(S)-4-carboxy-4-((S)-4-carboxy-4-{6-[4-(16-(1H-tetrazol-5-yl)hexadecanoylsulfamoyl) butyrylamino]hexanoylamino}butyrylamino) butyrylamino]ethoxy}ethoxy)acetyl][Aib8,Arg34]GLP-1-(7-35); N-epsilon26-[2-(2-{2-[(S)-4-carboxy-4-((S)-4-carboxy-4-{12-[4-(16-(1H-tetrazol-5-yl)hexadecanoyl-sulfamoyl)butyrylamino]dodecanoylamino}butyryl-amino)butyrylamino]ethoxy}ethoxy)acetyl][Aib8,Lys33,Arg34]GLP-1-(7-34); N-epsilon26-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyrylamino)butyrylamino]ethoxy}ethoxy)acetyl][Aib8,Arg34]GLP-1-(7-36)amide; N-epsilon26-[2-(2-{2-[2-(2-{2-[2-(2-{2-[2-(2-{2-[2-(2-{2-[2-(2-{2-[(S)-4-carboxy-4-((S)-4-carboxy-4-{12-[4-(16-(1H-tetrazol-5-yl)hexadecanoylsulfamoyl) butyrylamino]dodecanoylamino}butyrylamino) butyrylamino]ethoxy}ethoxy) acetylamino]ethoxy}ethoxy)acetylamino]ethoxy}ethoxy)acetylamino]ethoxy}ethoxy)acetylamino]ethoxy}ethoxy)acetylamino]ethoxy}ethoxy)acetyl][Aib8,Lys26,Arg34]GLP-1-(7-36)amide; N-epsilon37-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyrylamino) butyrylamino]ethoxy}ethoxy)acetyl][Aib8,Glu22,Arg26,Arg34,Lys37]GLP-1-(7-37)amide; N-epsilon 37-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyrylamino) butyrylamino]ethoxy}ethoxy)acetyl][DesaminoHis7,Glu22,Arg26,Arg34,Lys37]GLP-1-(7-37)amide; N-epsilon 37{2-[2-(2-{2-[2-((R)-3-carboxy-3-{[1-(19-carboxy-nonadecanoyl) piperidine-4-carbonyl]amino}propionylamino)ethoxy]ethoxy}acetylamino)ethoxy]ethoxy}acetyl[desaminoHis7,Glu22,Arg26,Arg34,Lys37]GLP-1(7-37)amide; N-epsilon37{2-[2-(2-{2-[2-((S)-3-carboxy-3-{[1-(19-carboxynonadecanoyl) piperidine-4-carbonyl]amino}propionylamino) ethoxy]ethoxy}acetylamino)ethoxy]ethoxy}acetyl[Aib8,Glu22, Arg26,Arg34, Lys37]GLP-1-(7-37)amide; N-epsilon37-[2-(2-[2-(2-[2-(2-((R)-3-[1-(17-carboxyhepta-decanoyl)piperidin-4-ylcarbonylamino]3-carboxy-propionylamino) ethoxy)ethoxy]acetylamino) ethoxy]ethoxy)acetyl][DesaminoHis7, Glu22,Arg26, Arg34,Phe(m-CF3)28]GLP-1-(7-37)amide; N-epsilon 37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetylamino]ethoxy}ethoxy)acetyl][Aib8,Glu22,Arg26,Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexane-carbonyl}amino)butyrylamino]ethoxy}ethoxy) acetylamino]ethoxy}ethoxy)acetyl][DesaminoHis7,Glu22,Arg26,Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy) acetylamino]ethoxy}ethoxy)acetyl][DesaminoHis7,Glu22,Arg26,Arg34, Lys37]GLP-1-(7-37); N-epsilon37-[2-(2-{2-[2-(2-{2-[(S)-4-carboxy-4-({trans-4-[(19-carboxy-nonadecanoylamino)methyl]cyclohexane-carbonyl}amino)butyrylamino]ethoxy}ethoxy) acetylamino]ethoxy}ethoxy)acetyl][DesaminoHis7,Glu22,Arg26,Glu30,Arg34, Lys37]GLP-1-(7-37); N-epsilon 37-[2-(2-{2-[(S)-4-carboxy-4-((S)-4-carboxy-4-{12-[4-(16-(1H-tetrazol-5-yl)hexadecanoyl-sulfamoyl) butyrylamino]dodecanoylamino}butyrylamino) butyrylamino]ethoxy}ethoxy)acetyl][Aib8,Glu22,Arg26,Arg34,Lys37]GLP-1-(7-37)amide; N-epsilon37-[2-(2-{2-[(S)-4-carboxy-4-((S)-4-carboxy-4-{12-[4-(16-(1H-tetrazol-5-yl)hexadecanoylsulfamoyl) butyrylamino]dodecanoylamino}butyrylamino) butyrylamino]ethoxy}ethoxy)acetyl][DesaminoHis7,Glu22,Arg26,Arg34,Lys37]GLP-1-(7-37)amide; N-epsilon37-(3-((2-(2-(2-(2-(2-Hexadecyloxyethoxy)ethoxy)ethoxy) ethoxy) ethoxy)) propionyl)[DesaminoHis7,Glu22,Arg26,Arg34,Lys37]GLP-1(7-37)-amide; N-epsilon37-{2-(2-(2-(2-[2-(2-(4-(hexadecanoylamino)-4-carboxybutyryl-amino)ethoxy) ethoxy]acetyl)ethoxy)ethoxy)acetyl)}-[desaminoHis7,Glu22,Arg26, Glu30,Arg34,Lys37]GLP-1-(7-37)amide; N-epsilon37-{2-(2-(2-(2-[2-(2-(4-(hexadecanoylamino)-4-carboxy-butyryl-amino) ethoxy)ethoxy]acetyl)ethoxy)ethoxy) acetyl)}-[desaminoHis7,Glu22, Arg26, Arg34,Lys37]GLP-1-(7-37)amide; N-epsilon37-(2-(2-(2-(2-(2-(2-(2-(2-(2-(octadecanoyl-amino)ethoxy)ethoxy) acetylamino)ethoxy) ethoxy)acetylamino) ethoxy)ethoxy) acetyl)[desaminoHis7,Glu22,Arg26,Arg34,Lys37]GLP-1 (7-37)amide; N-epsilon37-[4-(16-(1H-Tetrazol-5-yl)hexadecanoylsulfamoyl) butyryl][DesaminoHis7,Glu22,Arg26, Arg34, Lys37]GLP-1-(7-37)amide; N-epsilon37-[2-(2-{2-[2-(2-{2-[(S)-4-carboxy-4-(19-carboxynonadecanoylamino) butyrylamino]ethoxy}ethoxy) acetylamino]ethoxy}ethoxy)acetyl][DesaminoHis7,Glu22,Arg26, Arg34,Lys37]GLP-1-(7-37); N-epsilon37-(2-{2-[2-((S)-4-carboxy-4-{(S)-4-carboxy-4-[(S)-4-carboxy-4-(19-carboxy-nonadecanoylamino)butyrylamino]butyrylamino}butyrylamino)ethoxy]ethoxy}acetyl)[DesaminoHis7,Glu22,Arg26,Arg34,Lys37]GLP-1-(7-37); N-epsilon37-{2-[2-(2-{(S)-4-[(S)-4-(12-{4-[16-(2-tert-Butyl-2H-tetrazol-5-yl)-hexadecanoylsulfamoyl]butyrylamino}dodecanoylamino)-4-carboxybutyrylamino]-4-carboxybutyrylamino}ethoxy)ethoxy]acetyl}[DesaminoHis7,Glu22,Arg26,Arg34,Lys37]GLP-1 (7-37); N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl][Aib8,Glu22, Arg26,Arg34,Lys37]GLP-1-(7-37); N-alpha37-[2-(2-{2-[2-(2-{2-[(S)-4-carboxy-4-(17-carboxy-heptadecanoylamino)- butyrylamino]-ethoxy}-ethoxy)-acetylamino]ethoxy}-ethoxy)-acetyl][Aib8,Glu22,Arg26,Arg34,epsilon-Lys37]GLP-1-(7-37)peptide; N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl][desaminoHis7, Glu22,Arg26,Arg34,Lys37]GLP-1-(7-37); N-epsilon36-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetyl][desaminoHis7, Glu22,Arg26,Glu30,Arg34,Lys36]GLP-1-(7-37)-Glu-Lys peptide; N-epsilon37-[2-(2-{2-[2-(2-{2-[(S)-4-carboxy-4-({trans-4-[(19-carboxynonadecanoylamino)methyl]cyclohexanecarbonyl}amino)butyryl-amino]ethoxy}ethoxy)acetylamino]ethoxy}ethoxy)acetyl][Aib8,Glu22,Arg26,Arg34,Lys37]GLP-1-(7-37); N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl]-[Aib8,Glu22, Arg26,Arg34,Aib35,Lys37]GLP-1-(7-37); N-epsilon37-[(S)-4-carboxy-4-(2-{2-[2-(2-{2-[2-(17-carboxyheptadecanoylamino)ethoxy]ethoxy}acetylamino) ethoxy]ethoxy}acetylamino) butyryl][Aib8,Glu22,Arg26,34,Lys37]GLP-1 (7-37); N-epsilon37-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][ImPr7,Glu22, Arg26,34,Lys37], GLP-1-(7-37); N-epsilon26-{2-[2-(2-{2-[2-(2-{(S)-4-carboxy-4-[10-(4-carboxyphenoxyl) decanoylamino]butyrylamino}ethoxy)ethoxy]acetylamino}ethoxy)ethoxy]acetyl}, N-epsilon 37-{2-[2-(2-{2-[2-(2-{(S)-4-carboxy-4-[10-(4-carboxy-phenoxy) decanoylamino]butyrylamino}ethoxy)ethoxy]acetylamino}ethoxy) ethoxy]acetyly}-[Aib8,Arg34,Lys37]GLP-1(7-37)-OH; N-epsilon26 (17-carboxyhepta-decanoyl)-[Aib8,Arg34]GLP-1-(7-37)-peptide; N-epsilon 26-(19-carboxynonadecanoyl)-[Aib8,Arg34]GLP-1-(7-37); N-epsilon26-(4-{[N-(2-carboxyethyl)-N-(15-carboxypenta-decanoyl)amino]methyl}benzoyl[Arg34]GLP-1-(7-37); N-epsilon 26-[2-(2-[2-(2-[2-(2-[4-(17-carboxyheptadecanoylamino)-4(S)-carboxybutyrylamino]ethoxy)ethoxy]acetylamino) ethoxy]ethoxy)acetyl][Aib8,Arg34]GLP-1-(7-37); N-epsilon26-[2-(2-[2-(2-[2-(2-[4-(19-carboxynonadecanoylamino)-4(S)-carboxybutyrylamino]ethoxy)ethoxy]acetylamino)ethoxy]ethoxy)acetyl][Aib8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][3-(4-Imidazolyl)Propionyl7,Arg34]GLP-1-(7-37); N-epsilon26-[2-(2-[2-(2-[2-(2-[4-(17-carboxyheptadecanoylamino)-(carboxymethyl-amino)acetylamino]ethoxy)ethoxy]acetylamino)ethoxy]ethoxy)acetyl][Aib8,Arg34]GLP-1-(7-37); N-epsilon26-[2-(2-[2-(2-[2-(2-[4-(17-carboxyheptadecanoylamino)-3(S)-Sulfopropionylamino]ethoxy)ethoxy]acetylamino)ethoxy]ethoxy)acetyl][Aib8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Gly8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Aib8,Arg34]GLP-1-(7-37)-amide; N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Aib8,Arg34,Pro37]GL P-1-(7-37)amide; Aib8,Lys26(N-epsilon26-{2-(2-(2-(2-[2-(2-(4-(pentadecanoylamino)-4-carboxybutyrylamino)ethoxy)ethoxy]acetyl)ethoxy) ethoxy)acetyl)}), Arg34)GLP-1H(7-37)-OH; N-epsilon26-[2-(2-[2-(2-[2-(2-[4-{[N-(2-carboxyethyl)-N-(17-carboxyheptadecanoyl)amino]methyl}benzoyl)amino]ethoxy) ethoxy]acetylamino)ethoxy]ethoxy)acetyl][Aib8,Arg34]GLP-1(7-37); N-alpha7-formyl, N-epsilon 26-[2-(2-[2-(2-[2-(2-[4-(17-carboxyheptadecanoyl-amino)-4(S)-carboxybutyrylamino]ethoxy)ethoxy]acetylamino)ethoxy]ethoxy)acetyl][Arg34]GLP-1-(7-37); N-epsilon 2626-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Aib8, Glu22, Arg34]GLP-1-(7-37); N-epsilon26{3-[2-(2-{2-[2-(2-{2-[2-(2-[4-(15-(N—((S)-1,3-dicarboxypropyl) carbamoyl)pentadecanoylamino)-(S)-4-carboxybutyrylamino]ethoxy)ethoxy]ethoxy}ethoxy)ethoxy]ethoxy}ethoxy)ethoxy]propionyl}[Aib8,Arg34]GLP-1-(7-37); N-epsilon 26-[2-(2-[2-(2-[2-(2-[4-{[N-(2-carboxyethyl)-N-(17-carboxy-heptadecanoyl)amino]methyl}benzoyl)amino](4(S)-carboxybutyryl-amino)ethoxy) ethoxy]acetylamino)ethoxy]ethoxy)acetyl][Aib8,Arg34]GLP-1(7-37); N-epsilon26-{(S)-4-carboxy-4-((S)-4-carboxy-4-((S)-4-carboxy-4-((S)-4-carboxy-4-(19-carboxy-nonadecanoylamino)butyrylamino)butyrylamino)butyrylamino) butyrylamino}[Aib8,Arg34]GLP-1-(7-37); N-epsilon26-4-(17-carboxyheptadecanoyl-amino)-4(S)-carboxybutyryl-[Aib8,Arg34]GLP-1-(7-37); N-epsilon26-{3-[2-(2-{2-[2-(2-{2-[2-(2-[4-(17-carboxyheptadecanoylamino)-4(S)-carboxybutyrylamino]ethoxy)ethoxy]ethoxy}ethoxy)ethoxy]ethoxy}ethoxy)ethoxy]propionyl}[Aib8,Arg34]GLP-1-(7-37); N-epsilon26-{2-(2-(2-(2-[2-(2-(4-(17-carboxyheptadecanoylamino)-4-carboxybutyrylamino) ethoxy)ethoxy]acetyl)ethoxy)ethoxy)acetyl)}-[Aib8,22,27,30,35,Arg34,Pro37, Lys26]GLP-1 (7-37)amide; N-epsilon26-[2-(2-[2-[4-(21-carboxyuneicosanoylamino)-4(S)-carboxybutyrylamino]ethoxy]ethoxy)acetyl][Aib8,Arg34]GLP-1-(7-37); and N-epsilon26-[2-(2-[2-(2-[2-(2-[4-(21-carboxyuneicosanoylamino)-4(S)-carboxybutyrylamino]ethoxy)ethoxy]acetylamino)ethoxy]ethoxy)acetyl][Aib8,Arg34]GLP-1-(7-37). In one embodiment the GLP-1 peptide is N-epsilon26-{2-[2-(2-{2-[2-(2-{(S)-4-carboxy-4-[10-(4-carboxyphenoxyl)decanoylamino]butyrylamino}ethoxy)ethoxy]acetylamino}ethoxy) ethoxy]acetyl}, N-epsilon37-{2-[2-(2-{2-[2-(2-{(S)-4-carboxy-4-[10-(4-carboxy-phenoxy) decanoylamino]butyrylamino}ethoxy)ethoxy]acetylamino}ethoxy) ethoxy]acetyl}-[Aib8,Arg34,Lys37]GLP-1(7-37)-OH (Compound A).

In certain embodiments, the drug is present in a therapeutically effective amount. The term “therapeutically effective amount” (dose) of the drug refers to an amount of the drug that, when administered to a subject/patient to treat a disease, is sufficient to effect treatment for the disease. The “therapeutically effective amount” of each drug can vary depending on the type of disease and its severity, and the age, weight, etc., of the subject to be treated.

In certain embodiments, the drug is present in the range of 0.01 mg to 500 mg, such as in the range of at least 0.5 mg, at least 1 mg, at least 5 mg, at least 10 mg, at least 15 mg, at least 20 mg, at least 25 mg, or at least 100 mg.

In some embodiments, the pharmaceutical formulation comprises the acid neutralizer in the amount of about 0.1-80%, such as 1-75%, 2-65%, 2.5-50%, 3-40%, 3.5-35% (w/w) of the total amount of the pharmaceutical formulation. In some embodiments the pharmaceutical formulation comprises the acid neutralizer in an amount of more than about 0.1%, such as >1%, >1.5%, >2.5%, >5%, >10%, >15%, >20%, >25%, >30%, but less than about of 80%, such as <75%, <65%, <55%, <50%, <40% (w/w) of the total amount of the pharmaceutical formulation. In certain embodiments, the acid neutralizer includes, but is not limited to, an alkali or an earth alkali metal salt, alkaline oxide, alkaline hydroxides, alkaline carbonates, alkaline phosphates, basic amino acids and meglumine. In certain embodiments, the acid neutralizer includes, but is not limited to, magnesium oxide, meglumine, sodium oxide, sodium hydroxide, sodium bicarbonate, sodium potassium tartrate, bismuth aluminate, bismuth carbonate, bismuth subcarbonate, bismuth subgallate, bismuth subnitrate, potassium citrate, sodium carbonate, potassium bicarbonate, potassium carbonate, calcium carbonate, calcium phosphate, dibasic calcium phosphate, dihydroxyaluminumaminoacetate, dihydroxyaluminum sodium carbonate, glycine, magnesium glycinate, magnesium hydroxide, magnesium carbonate, sodium borate, aluminum oxide, aluminum hydroxide, ammonium carbonate, monoethanolamide, diethanolamine, triethanolamine, potassium hydroxide, calcium hydroxide, sodium phosphate dibasic, trolamine, sodium potassium tartrate, tribasic sodium phosphate, tricalcium phosphate, any combination thereof, and like. In certain embodiments, the acid neutralizer further includes antacids or agents that inhibit acid secretion in the stomach, such as, but not limited to, histamine receptor antagonists and proton pump inhibitors. In certain embodiments, the acid neutralizer is present in an amount ranging from about 10 mg to 1000 mg per dosage unit, such as from about 15 mg to about 750 mg, from about 20 mg to 500 mg, from about 25 mg to 400 mg, from about 30 mg to 300 mg per dosage unit or composition.

In some embodiments, the pharmaceutical formulation comprises the alkaline salt of an oral absorption promoter in an amount of about 1-99%, such as 5-98%, 10-96%, 15-95%, 30-90%, 35-85% (w/w) of the total amount of the pharmaceutical formulation. In some embodiments the pharmaceutical formulation comprises the alkaline salt of an oral absorption promoter in an amount of more than about 1%, such as >2.5%, >5%, >7.5%, >10%, >15%, >20%, >25%, >30%, >35%, but less than about of 99%, such as <98%, <96%, <95%, <90%, <85% (w/w) of the total amount of the pharmaceutical formulation.

In certain embodiments, an oral absorption promoter is neutralized using any alkali/alkaline material to form the alkaline salt of an oral absorption promoter. In certain embodiments, the alkaline salt includes, for examples, but is not limited to, lithium, sodium, potassium, calcium, magnesium, ammonium or alkanolammonium, triethylammonium, or triethanolammonium salt. In most certain embodiments, the alkaline salt is sodium or potassium salt. However, the type of alkali/alkaline material to form the alkaline salt of an oral absorption promoter does not limit the scope of the present invention.

In certain embodiments, the alkaline salt of oral absorption promoter includes an alkaline salt of at least one selected from the group consisting of, but not limited to, caproic acid (also known as hexanoic acid), caprylic acid (also known as octanoic acid), capric acid (also known as decanoic acid or dicyclic acid) such as sodium caprate, nipecotic acid, butyric acid (also known as butanoic acid), amino acid, propionic acid, hydroxamic acid, succinic acid, nicotinic acid, valeric acid (also known as pentanoic acid), nonanoic acid (also known as pelargonic acid), sebacic acid, sebalic acid, heptanoic acid (also known as enanthic acid), carboxylic acid, acetic acid, or any derivative thereof.

In certain embodiments, the pharmaceutical formulation further comprises an oral absorption promoter. In some embodiments, the pharmaceutical formulation comprises the oral absorption promoter in amount of about 1-99%, such as 5-98%, 10-96%, 15-95%, 30-90%, 35-85% (w/w) of the total amount of the pharmaceutical formulation. In some embodiments the pharmaceutical formulation comprises the oral absorption promoter in an amount of more than about 1%, such as >2.5%, >5%, >7.5%, >10%, >15%, >20%, >25%, >30%, >35%, but less than about of 99%, such as <98%, <96%, <95%, <90%, <85% (w/w) of the total amount of the pharmaceutical formulation.

In certain embodiments, the oral absorption promoter or the alkaline salt of the oral absorption promoter includes at least one selected from the group consisting of, but not limited to, 8-(salicyloylamino)octanoic acid, 10-(salicyloyiamino)decanoic acid, 6-N-(3,5-dichloro-2-hydroxybenzoyl)aminocaproic acid, 8(2-aminobenzoylamino)caprylic acid, 8(2-trifluoromethoxy)benzoylamino caprylic acid, N-(2-hydroxybenzoyl)isonipecotic acid, 4-[4-(2-aminobenzoylamino)phenyl]butyrylhydroxamic acid, 4-(4-(pentafluorobenzoyl)aminophenyl)butyric acid, 4-(4-(3-anisoyl)aminophenyl)butyric acid, 8-(3-anisoyl)aminocaprylic acid, 4-(4-(phenoxyacetyl)aminophenyl)butyric acid, 4-(4-(2-nitrobenzenesulfonyl)aminophenyl)butyric acid, 8-(2-nitrobenzenesulfonyl)amino caprylic acid, 6-(4-(salicyloyl)aminophenyl)hexanoic acid, 8-(2-methoxybenzoyl)amino caprylic acid, 2-[4-Salicyloylamino)phenyl]ethyl methyl sulfone, 1-Salicyloyl-2-succinyl hydrazide, 3-(4-(2,5-dimethoxycinnamoyl)aminophenyl)propionic acid, 4-(4-(2,5-dimethoxycinnamoyl)aminophenyl)butyric acid, 1-salicyloyl-2-glutaryl hydrazide, Succinyl-4-aminosalicyclic acid, 8-(Phenoxyacetylamino)caprylic acid, 8-(2-pyrazinecarbonyl)aminocaprylic acid, 4-(4-(2-pyrazinecarbonyl)aminophenyl)butyric acid, 6-(4-(N-2-Nitrobenzoyl)aminophenyl)hexanoic acid, 6-(4-(N-2-aminobenzoyl)aminophenyl)hexanoic acid, 4-(4-(2-(3-carboxyl)pyrazinecarboxyl)aminophenyl)butyric acid, 4(2-Nitrobenzoyl)aminophenylsuccinic acid, 8-(2-(trifluoromethoxy)benzoyl)aminocaprylic acid, 8-(Benzoylcarbonyl)caprylic acid, 8-(phenylcarbonylamino)caprylic acid, 2-[4-(2-Methoxybenzoylamino)phenyl]ethyl H₂PO₄, 1-salicyloyl-2-suberyl hydrazide, 4-(4-benzyloxycarbonylaminophenyl)butyric acid, 4-(4-(2-hydroxynicotinoyl)aminophenyl)butyric acid, 9-Salicyloylaminononanionic acid, 4-(4-phenyloxycarbonylaminophenyl)butyric acid, 8-(2-Hydroxynicotinoyl)aminocaprylic acid, 6-(2-methoxybenzoyl)amino nicotinic acid, salicyloylglycine, 4-(1-(2-pyrimidyl)piperazinoyl)butyric acid, 8-(chromone-3-carbonyl)aminocaprylic acid, 8-(vinylbenzoyl)aminocaprylic acid, 4-(4-(chromone-3-carbonyl)aminophenyl)butyric acid, 8-cinnamoylaminocapyrlic acid, 6-(N-salicyloylamino)valeric acid, 9-(2-hydroxybenzamido)nonanic acid, N-(4-salicyloylamino)-6-caproic acid, 11-cinnamoylaminoundecanoic acid, 4′-flavonic acid, 4-octanoylamino-3-hydroxybenzoic acid, (3Phenyl2,3dihydroxypropanoyl)8aminocaprylic acid, 8-[N-(3-coumarincarbonyl)]aminocaprylic acid, 8-[N-(4-chlorobenzoyl)]aminocaprylic acid, 8-[N-(3-fluorobenzoyl)]aminocaprylic acid, 8-(N-2,5-Dihydroxybenzoyl)aminocaprylic acid, 8-(N-2,3-Dimethoxybenzoyl)aminocaprylic acid, 8-(N-2,4-Dihydroxybenzoyl)aminocaprylic acid, 8-(N-2,5-Dimethoxybenzoyl)aminocaprylic acid, 8-(N-3,5-Diacetyloxybenzoyl)aminocaprylic acid, 8-(N-4-Hydroxybenzoyl)aminocaprylic acid (dimer), 8-(N-2,4-Dihydroxybenzoyl)aminocaprylic acid, 10(N-2-Methoxyanilino)sebalic acid, 10(N-2-Methoxyanilino)sebacic acid, 2-Methoxybenzeneaminodecanoic acid, 8-(N-benzoyl)aminocaprylic acid, 8-(N-2-Hydroxy-4-methoxybenzoyl)aminocaprylic acid, 8-[N-(4-fluorobenzoyl)aminocaprylic acid, 8-[N-(3-bromobenzoyl)]aminocaprylic acid, 8-(4-(1,2-dihydroxyethyl)benzoyl)aminocaprylic acid, 8-[N-(4-bromobenzoyl)]aminocaprylic acid, 8-[N-(4-iodobenzoyl)]aminocaprylic acid, 4-{4-[N-(2-iodobenzoyl)aminophenyl]}butyric acid, 4-{4-[N-(1-hydroxy-2-naphthoyl)aminophenyl]}butyric acid, 4-(4-(2,4-dimethoxybenzoyl)aminophenyl)butyric acid, 4-(o-anisoyl)aminophenylacetic acid, 3-[4-(2,4-dimethoxybenzoyl)aminophenyl]propionic acid, 4-{4-[N-(4-iodobenzoyl)]aminophenyl}butyric acid, 3-[4-(2,3-dimethoxybenzoyl)aminophenyl]propionic acid, 4{4-[N-2-bromobenzoyl)]aminophenyl}butyric acid, 4{4-[N-3-bromobenzoyl)aminophenyl}butyric acid, 8-(N-3,5-Dihydroxybenzoyl)aminocaprylic acid, 8-(N-3,5-Dimethoxy-4-hydroxybenzoyl)aminocaprylic acid, 8-(N-2-6-Dimethoxybenzoyl)aminocaprylic acid, 4-{4-[N-(4-bromobenzoyl)aminophenyl}butyric acid, 8-(2-hydroxy-4-chlorobenzoyl)aminocaprylic acid, 8-(N-2,6-Dihydroxybenzoyl)aminocaprylic acid, 8-(N-2-Hydroxy6-methoxybenzoyl)aminocaprylic acid, 8-(5-chloro-o-anisoyl)aminocaprylic acid, 4-(4-(2,3-dimethoxybenzoyl)aminophenyl)butyric acid, 4-(4-(5-chloro-o-anisoyl)aminophenyl)butyric acid, 4-(4-(4-chloro-o-anisoyl)aminophenyl)butyric acid, 8-(4-chloro-o-anisoyl)aminocaprylic acid, 3-(4-(2,5-dimethoxybenzoyl)aminophenyl)propionic acid, 4-{N-[4-(3-iodobenzoyl)aminophenyl}butyric acid, 7-cinnamoylaminoheptanoic acid, 8-N-(3-iodobenzoyl)aminocaprylic acid, 8-N-(4-methoxy-3-nitrobenzoyl)aminocaprylic acid, 8-N-(2-methoxy-4-nitrobenzoyl)aminocaprylic acid, 4-{N-[4-(2-methoxy-4-nitrobenzoyl)aminophenyl}butyric acid, 4-(4-(2,5-dimethoxybenzoyl)aminophenyl)butyric acid, 8-(N-2-hydroxy-5-bromobenzoyl)aminocaprylic acid, 3-Indolebutyric acid, 4-(4-(2,6-dimethoxybenzoyl)aminophenylbutyric acid, 4-[4-N-(4-methoxy-3-nitrobenzoyl)aminophenyl]butyric acid, 8-(N-2-hydroxy-5-chlorobenzoyl)aminocaprylic acid, 8-(N-2-hydroxy-5-iodobenzoyl)aminocaprylic acid, 8-(3-Phenoxy)propionylamino)caprylic acid, 8-(N-2-hydroxy-4-nitrobenzoyl)aminocaprylic acid, 4-[—N-(2-hydroxy-4-bromobenzoyl)aminophenyl]butyric acid, 8-(N-2,3-Dihydroxybenzoyl)aminocaprylic acid, 8-(N-3-methylsalicyloyl)aminocaprylic acid, 8-(N-5-methylsalicyloyl)aminocaprylic acid, 9-(cinnamoylamino)nonanionic acid, 4-(4-(2-chloro-5-nitrobenzoyl)aminophenyl)butyric acid, 4-{-[N-(2-hydroxy-5-iodobenzoyl)]aminophenyl}butyric acid, 8-[N-(2-acetoxy-3,5-dichlorobenzoyl)]aminocaprylic acid, 8-[N-(2-acetoxy-3,5-dibromobenzoyl)]aminocaprylic acid, 8-N-(2-chloro-6-fluorobenzoyl)aminocaprylic acid, 8-N-(4-hydroxy-3-nitrobenzoyl)caprylic acid, 4-(4-Salicyloylaminophenyl)-4-oxobutyric acid, 12-cinnamoyldodecanoic acid, 4-{4-[N-(3-hydroxy-2-naphthoyl)aminophenyl]}butyric acid, 8-(4-chloro-3-nitrobenzoyl)aminocaprylic acid, 8-(2-chloronicotinoyl)aminocaprylic acid, 8-(2-chloro-5-nitrobenzoyl)aminocaprylic acid, 4-(4-phthalimidophenyl)butyric acid, 4-{4-[N-(3-hydroxy-2-naphthoyl)aminophenyl]}propanoic acid, 3-(4-(2,6-dimethoxybenzoyl)aminophenyl)propionic acid, 8-(N-2-hydroxy-3,5-diiodobenzoyl)aminocaprylic acid, 8-(N-2-chloro-4-fluorobenzoyl)aminocaprylic acid, 8-(2-(1,2-dihydroisoindole-1-one)octanoic acid, 8-(N-1-hydroxy-2-naphthoyl)aminocaprylic acid, 8-(phthalimido)caprylic acid, 10(4-chloro-2-hydroxyanilino)sebaic acid monoamide, 4-(4-(4-chloro-3-nitrobenzoyl)aminophenyl)butyric acid, 11-N-(1-hydroxy-2-naphthoyl)aminoundecanoic acid, 4-(4-(3-hydroxyphthalimido)phenyl)butyric acid, trans-4-(2-aminobenzamidomethyl)cyclohexamecarboxylic acid, 11-N-(3,5-dichloro-2-hydroxybenzoyl)aminoundecanoic acid, 7-N-(3,5-dichloro-2-hydroxybenzoyl)aminoheptanoic acid, N-[3,5-dichloro-2-hydroxybenzoyl-4(4-aminophenyl)]butyric acid, trans-4-(N-salicyloylaminomethyl)cyclohexane carboxylic acid, N-[3,5-dichloro-2-hydroxybenzoyl-3-(4-aminophenyl)]propionic acid, 12-N-(3,5-dichloro-2-hydroxybenzoyl)aminododecanoic acid, 8-N-cyclohexanoylaminocaprylic acid, 5-(4-chloro-2-hydroxyanilinocarbonyl)valeric acid, 8-(2-hydroxyphenoxy)-aminocaprylic acid, N-Salicyloyl-5-(3-aminophenyl)valeric acid, 4-(4-(2-ethoxybenzoyl)aminophenyl)butyric acid, 9-[2-(3-hydroxy)pyridylaminocarbonyl]nonanic acid, 7-(2-hydroxyphenoxyacetyl)aminocaprylic acid, 4-[N-(3,5-dichloro-2-hydroxybenzoyl)]aminophenylacetic acid, 8-(2-hydroxy-5-chloroanilinocarbonyl)octanoic acid, N-salicyloyl-5-(4-aminophenyl)valeric acid, 9-(2-hydroxy-5-methylanilinocarbonyl)nonanoic acid, 5-(2-hydroxy-5-methylanilinocarbonyl)valeric acid, 8-(pentafluorobenzoyl)aminocaprylic acid, 3-(3-(salicyloyl)aminophenyl)propionic acid, 8-(2-ethoxybenzoyl)aminocaprylic acid, 4-(4-(2-Diethylaminobenzoic)aminophenyl)butyric acid, 8-(3-Phenoxy)propionylamino)caprylic acid, 8(-(4(N-Salicyloyl-4aminophenyl) butyric)aminocaprylic acid, 4-(4-(N-(2-Fluorocinnamoyl))aminophenyl) butyric acid, 4-(4-(N-8(N-Salicyloyl)aminocaprylic)aminophenyl) butyric acid, 8-(p-anisoyl)aminocaprylic acid, 8-(4-Hydroxybenzoyl)aminocaprylic acid, 8-(3-Hydroxybenzoyl)aminocaprylic acid, 8-(3,4,5-Trimethoxybenzoyl)aminocaprylic acid, 8-(N-4-Methylsalicyloyl)aminocaprillic acid [sic], N-10-(2-hydroxy-5-nitroanilino)decanoic acid, 4-(4-(2-chloronicotinoyl)aminophenyl)butyric acid, and 6-(anisoyl)aminocaprylic acid.

In certain embodiments, the oral absorption promoter or the alkaline salt of the oral absorption promoter includes any of the compounds, ingredients, or carriers which are mentioned in patents (or patent applications) U.S. Pat. Nos. 8,686,154B2, 5,650,386A, 10,960,052B2, and EP3006045B1, the entire patents herein incorporated as reference.

In certain embodiments, the oral absorption promoter includes, but is not limited to, at least one selected from the group consisting of a zwitter-ionic absorption enhancer or a non-ionic absorption enhancer. In certain embodiments, the oral absorption enhancer or the alkaline salt of the oral absorption promoter is selected from any or a combination of C8-20 alkanoyl carnitine (preferably lauroyl carnitine, myristoylcarnitine or palmitoyl carnitine; e.g., lauroyl carnitine chloride, myristoyl carnitine chloride or paimitoyi carnitine chloride), a salicylic acid derivative (such as 3-methoxysalicylic acid, 5-methoxysalicylic acid, or homovanillic acid, a C8-20 alkanoic acid (preferably a C8-20 alkanoate, more preferably a caprate, a caprylate, a myristate, a palmitate, or a stearate, such as sodium caprate, sodium caprylate, sodiummyristate, sodium palmitate, or sodium stearate), a fatty acid acylatedamino acid (any of the fatty acid acylated amino acids disclosed in US patent application US20140056953A1 without being limited thereto, sodium lauroyl alaninate, N-dodecanoyl-L-alanine, sodiumlauroyl asparaginate, N-dodecanoyl-L-asparagine, sodium lauroyl aspartic acid, N-dodecanoyl-L-aspartic acid, sodiumlauroyl cysteinate, N-dodecanoyl-L-cysteine, sodium lauroyl glutamic acid, N-dodecanoyl-L-glutamic acid, sodium lauroylglutaminate, N-dodecanoyl-L-glutamine, sodium lauroyl glycinate, N-dodecanoyl-L-glycine, sodium lauroyl histidinate,N-dodecanoyl-L-histidine, sodium lauroyl isoleucinate, N-dodecanoyl-L-isoleucine, sodium lauroyl leucinate, N-dodecanoyl-L-leucine, sodium lauroyl methioninate, N-dodecanoyl-L-methionine, sodium lauroyl phenylalaninate, N-dodecanoyl-L-phenylalanine, sodium lauroyl prolinate, N-dodecanoyl-L-proline, sodium lauroyl serinate, N-dodecanoyl-Lserine,sodium lauroyl threoninate, N-dodecanoyl-L-threonine, sodium lauroyl tryptophanate, N-dodecanoyl-L-tryptophane,sodium lauroyl tyrosinate, N-dodecanoyl-L-tyrosine, sodium lauroyl valinate, N-dodecanoyl-L-valine, sodiumlauroyl sarcosinate, N-dodecanoyl-L-sarcosine, sodium capric alaninate, N-decanoyl-L-alanine, sodium capric asparaginate,N-decanoyl-L-asparagine, sodium capric aspartic acid, N-decanoyl-L-aspartic acid, sodium capric cysteinate,N-decanoyl-L-cysteine, sodium capric glutamic acid, N-decanoyl-L-glutamic acid, sodium capric glutaminate, N-decanoyl-L-glutamine, sodium capric glycinate, N-decanoyl-L-glycine, sodium capric histidinate, N-decanoyl-L-histidine,sodium capric isoleucinate, N-decanoyl-L-isoleucine, sodium capric leucinate, N-decanoyl-L-leucine, sodium capric methioninate, N-decanoyl-L-methionine, sodium capric phenylalaninate, N-decanoyl-L-phenylalanine, sodium capric prolinate,N-decanoyl-L-proline, sodium capric serinate, N-decanoyl-L-serine, sodium capric threoninate, N-decanoyl-Lthreonine, sodium capric tryptophanate, N-decanoyl-L-tryptophane, sodium capric tyrosinate, N-decanoyl-L-tyrosine,sodium capric valinate, N-decanoyl-L-valine, sodium capric sarcosinate, N-decanoyl-L-sarcosine, sodium oleoyl sarcosinate, sodium N-decylleucine, sodium stearoyl glutamate (Amisoft HS-11 P), sodium myristoyl glutamate (Amisoft MS-11), sodium lauroyl glutamate (Amisoft LS-11), sodium cocoyl glutamate (Amisoft CS-11), sodiumcocoyl glycinate (Amilite GCS-11), sodium N-decyl leucine, sodium cocoyl glycine, sodium cocoyl glutamate, sodium lauroyl alaninate, N-dodecanoyl-L-alanine, sodium lauroyl asparaginate, N-dodecanoyl-L-asparagine, sodium lauroyl aspartic acid, N-dodecanoyl-L-aspartic acid, sodium lauroyl cysteinate, N-dodecanoyl-L-cysteine, sodium lauroyl glutamicacid, N-dodecanoyl-L-glutamic acid, sodium lauroyl glutaminate, N-dodecanoyl-L-glutamine, sodium lauroyl glycinate, N-dodecanoyl-L-glycine, sodium lauroyl histidinate, N-dodecanoyl-L-histidine, sodium lauroyl isoleucinate, N-dodecanoyl-L-isoleucine, sodium lauroyl leucinate, N-dodecanoyl-L-leucine, sodium lauroyl methinoninate, N-dodecanoyl-L-methionine, sodium lauroyl phenylalaninate, N-dodecanoyl-L-phenylalanine, sodium lauroyl prolinate, N-dodecanoyl-L-proline, sodium lauroyl serinate, N-dodecanoyl-L-serine, sodium lauroyl threoninate, N-dodecanoyl-L-threonine, sodiumlauroyl tryptophanate, N-dodecanoyl-L-tryptophane, sodium lauroyl tyrosinate, N-dodecanoyl-L-tyrosine, sodiumlauroyl valinate, N-dodecanoyl-L-valine, N-dodecanoyl-L-sarcosine, sodium capric alaninate, N-decanoyl-L-alanine, sodiumcapric asparaginate, N-decanoyl-L-asparagine, sodium capric aspartic acid, N-decanoyl-L-aspartic acid, Sodium capric cysteinate, N-decanoyl-L-cysteine, sodium capric glutamic acid, N-decanoyl-L-glutamic acid, sodium capricglutaminate, N-decanoyl-L-glutamine, sodium capric glycinate, N-decanoyl-L-glycine, sodium capric histidinate, N-decanoyl-L-histidine, sodium capric isoleucinate, N-decanoyl-L-isoleucine, sodium capric leucinate, N-decanoyl-L-leucine, sodium capric methioninate, N-decanoyl-L-methionine, sodium capric phenylalaninate, N-decanoyl-L-phenylalanine,sodium capric prolinate, N-decanoyl-L-proline, sodium capric serinate, N-decanoyl-L-serine, sodium capric threoninate,N-decanoyl-L-threonine, sodium capric tryptophanate, N-decanoyl-L-tryptophane, sodium capric tyrosinate, N-decanoyl-L-tyrosine, sodium capric valinate, N-decanoyl-L-valine, sodium capric sarcosinate, sodium oleoyl sarcosinate, and the pharmaceutically acceptable salts of any of the aforementioned compounds such as C8-20 alkanoyl sarcosinate (a lauroyl sarcosinate, such as sodium lauroyl sarcosinate) or one of the 20 standard proteinogenic.alpha.-amino acids that is acylated with a C.sub.8-20 alkanoic acid), an alkylsaccharide (C.sub.1-20 alkylsaccharide such as C.sub.8-10 alkylpolysaccharide like Multitrope™ 1620-LQ-(MV), or n-octyl-beta-D-glucopyranoside, or n-dodecyl-beta-D-maltoside), a cyclodextrine (.alpha.-cyclodextrin,.beta.-cyclodextrin,.gamma.-cyclodextrin, methyl-.beta.-cyclodextrin, hydroxypropyl.beta.-cyclodextrin, or sulfobutylether.beta.-cyclodextrin), sodium N-[8-(2-hydroxybenzoyl)amino]caprylate (SNAC), a thiomer (includes the thiomers that are disclosed in Laffleur F et al., Future Med Chem. 2012, 4, 2205-16), a calcium chelating compound (ethylenediaminetetraacetic acid (EDTA), ethylene glycol tetraacetic acid (EGTA), sodium citrate, or polyacrylic acid), cremophor EL (Kolliphor E L; CAS no. 61791-12-6), chitosan, N,N,N-trimethyl chitosan, benzalkonium chloride, bestatin, cetylpyridinium chloride, cetyltrimethylammonium bromide, a C.sub.2-20 alkanol (e.g., ethanol, decanol, lauryl alcohol, myristyl alcohol, or palmityl alcohol), a C.sub.8-20 alkenol (e.g., oleyl alcohol), a C.sub.8-20 alkenoic acid (e.g., oleic acid), dextran sulfate, diethyleneglycol monoethyl ether (transcutol), 1-dodecylazacyclo-heptan-2-one (Azone), ethyl caprylate, glyceryl monolaurate, lysophosphatidylcholine, menthol, a C.sub.8-20 alkylamine, a C.sub.8-20 alkenylamine (e.g., oleylamine), phosphatidylcholine, a poloxamer, polyethylene glycol monolaurate, polyoxyethylene, polypropylene glycol monolaurate, a polysorbate (polysorbate 80), a deoxycholate (sodium deoxycholate), sodium glycocholate, sodium glycodeoxycholate, sodium lauryl sulfate (SDS), a taurocholate (e.g., sodium taurocholate), a taurodeoxycholate (sodium taurodeoxycholate), sucrose laurate, a sulfoxide (a (C.sub.1-10 alkyl), (C.sub.1-10alkyl)-sulfoxide, such as, decyl methyl sulfoxide, or dimethyl sulfoxide), cyclopentadecalactone, 8-(N-2-hydroxy-5-chloro-benzoyl)-amino-caprylic acid (5-CNAC), dodecyl-2-N,N-dimethylamino propionate (DDAIP), D-.alpha.-tocopheryl polyethylene glycol-1000 succinate (TPGS), and pharmaceutically acceptable salts of the aforementioned compounds and the likes.

In certain embodiments, the intestinal enzyme inhibitor is at least one metal in the form of any or a combination of a salt thereof and a complex thereof, or a combination of (1) at least one metal in the form of any or a combination of a salt thereof and a complex thereof; and (2) at least one reducing agent.

In certain embodiments, the at least one metal is selected from any or a combination of, but not limited to, vanadium, chromium, manganese, copper, and zinc. In certain embodiments, the at least one metal is in form of any or a combination of a salt thereof and a complex thereof. In certain embodiments, the any of the salt of vanadium and the complex of vanadium is selected independently from a group including: vanadium (V) oxide, sodium vanadate, vanadium sulfate, vanadyl sulfate, vanadium biguanide, bis(maltolato) oxavandium (IV), vanadium acetate, vanadyl picolinate and vanadyl citrate. In certain embodiments, the any of the salt of chromium and the complex of chromium is selected independently from a group including: chromium picolinate, chromium polynicotinate, chromium nicotinate, chromium chloride and chromium acetate. In certain embodiments, the any of the salt of manganese and the complex of manganese is selected independently from a group including: manganese gluconate, manganese sulfate, potassium permanganate and manganese chloride. In certain embodiments, the any of the salt of copper and the complex of copper is selected independently from a group including: copper chloride, copper acetate, copper sulfate, copper carbonate, copper lysine complex, copper citrate, and copper gluconate. In certain embodiments, the any of the salt of zinc and the complex of zinc is selected independently from a group including: zinc sulfate, zinc chloride, zinc acetate, zinc oxide, zinc ascorbate, zinc caprylate, zinc gluconate, zinc stearate, and zinc carbonate.

In certain embodiment, the at least one metal in form of any or a combination of a salt thereof and a complex thereof is selected from, but not limited to, platinum, gold, and ruthenium, iron/ferrous, aluminum, calcium, cobalt, or any metal known to or appreciated by a person skilled in the art such as those mentioned in the periodic table of elements (https://pubchem.ncbi.nlm.nih.gov/periodic-table/), which can be utilized, alone or in combination with the reducing agent, to serve its intended purpose, i.e., to inactivate the intestinal enzyme as laid in the present disclosure, without departing from the scope and spirit of the present invention.

In certain embodiments, the at least one metal in form of any or a combination of a salt thereof and a complex thereof is present in an amount of less than about 50 wt %, less than about 35 wt %, less than about 20 wt %, and preferably less than about 5 wt % of the total weight of the pharmaceutical composition. In certain embodiments, the at least one metal in form of any or a combination of a salt thereof and a complex thereof is present in an amount of at least greater than about 0.05 wt %, at least greater than about 0.5 wt %, and preferably at least about 5 wt % of the total weight of the pharmaceutical composition. In certain embodiments, the at least one metal in form of any or a combination of a salt thereof and a complex thereof is present in any amount ranging from about 0.001 mg to about 250 mg per dosage unit or in any effective amount (preferable in an amount that does not show a systemic effect) to serve its intended purpose, i.e., to inactivate the intestinal enzyme as laid in the present disclosure, without departing from the scope and spirit of the present invention.

In certain embodiments, the at least one reducing agent is selected from any or a combination of ascorbic acid (preferably an ascorbate such as sodium ascorbate), reduced glutathione (GSH), cysteine, uric acid, a reducing sugar, a reducing monosaccharide, such as glucose; glyceraldehyde; or galactose, or a reducing disaccharide, such as lactose or maltose, mannitol, alpha-tocopherol, vitamin A, alpha-lipoic acid, dihydro-alpha-lipoic acid (DHLA), a thiol-bearing compound, a thiomer (includes thiomers Laffleur F et al., Future Med Chem, 2012, 4, 2205-16), and the likes. In certain embodiments, a mixture of two or more reducing agents, can be used, preferably, ascorbate and reduced glutathione. As used herein, the term “reducing sugar” refers to a sugar that has an open-chain form with an aldehyde group or a free hemiacetal group and can thus act as a reducing agent. The reducing sugar may be, e.g., a reducing monosaccharide (e.g., glucose, glyceraldehyde or galactose) or a reducing disaccharide (e.g., lactose or maltose). However, any or a combination of reducing agent(s), as known to or appreciated by a person skilled in the art, can be utilized to serve its intended purpose, as laid in the present disclosure, without departing from the scope and spirit of the present invention.

In certain embodiments, the pharmaceutical composition includes a reducing agent in an amount ranging from about 0.1 mg to about 1000 mg per unit dose or composition, such as from about 10 mg to about 500 mg per unit dose or composition, from about 20 mg to about 400 mg per unit dose or composition, from about 25 mg to about 300 mg per unit dose or composition, from about 30 mg to about 250 mg per unit dose or composition, and from about 35 mg to about 200 mg per unit dose or composition.

In some embodiments, the pharmaceutical formulation comprises at least one pharmaceutically acceptable excipient. The term “excipient” as used herein broadly refers to any component other than the drug, acid neutralizer, and alkaline salt of an oral absorption promoter. The excipient may be an inert substance and serve various purposes, e.g. as a delivery agent, vehicle, filler (also known as diluents), binder, lubricant, glidant, disintegrant, crystallization retarders, acidifying agent, preservative, antioxidant, buffering agent, chelating agent, complexing agents, surfactant agent, emulsifying and/or solubilizing agents, sweetening agents, wetting agents stabilizing agent, colouring agent, flavouring agent, and/or to improve administration, and/or absorption of the drug. A person skilled in the art may select one or more of the aforementioned excipients with respect to the particular desired properties of the solid or liquid oral dosage form by routine experimentation and without any undue burden. The amount of each excipient used may vary within ranges conventional in the art. For example, any of the previously discussed excipient can be used within range, depending on their functionality without any limitation, such as sweetening agents, binder, disintegrant or crystallization retarders in the range of about 0.1-25% (w/w) [such as 1-20%, 2-15%, 2.5-10% (w/w)], preservative, stabilizing agent, antioxidant, coloring agent or flavouring agent in the range of about 0.001-10% (w/w) [such as 0.01-5%, 0.05-5%, 0.1-2.5% (w/w)], chelating agent, lubricant or glidant in the range of about 0.1-10% (w/w) [such as 0.5-5%, 0.75-2.5%, 1-2% (w/w)], complexing agents, stabilizing agent, delivery agent, vehicle, filler or acidifying agent in the range of about 1-99% (w/w) [such as 2.5-90%, 5-75%, 10-65% (w/w)], and surfactant agent, emulsifying and/or solubilizing agents, or wetting agents in the range of about 0.1-25% (w/w) [such as 1-20%, 1.5-15%, 2-10% (w/w)]. Techniques and excipients which may be used to formulate oral dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

In certain embodiments, the fillers or diluents include one or more from cellulose derivatives, mono, di, or tri-basic calcium phosphate, sugar, carbohydrate, starch derivatives, acid or base, lactose (e.g. spray-dried lactose, α-lactose, β-lactose, Tabletose®, various grades of Pharmatose®, Microtose® or Fast-FloC®), microcrystalline cellulose (various grades of Avicel®, Elcema®, Vivacel®, Ming Tai® or Solka-Floc®), other cellulose derivatives, sucrose, sorbitol, mannitol, dextrins, dextrans, maltodextrins, dextrose, fructose, kaolin, mannitol, sorbitol, sucrose, sugar, starches or modified starches (including potato starch, maize starch and rice starch), calcium phosphate (e.g. basic calcium phosphate, calcium hydrogen phosphate, dicalcium phosphate hydrate), calcium sulphate, calcium carbonate, or sodium alginate. In some embodiments the filler is microcrystalline cellulose, such as Avicel PH 101 and like. Filler or diluent refers to material that is used to increase the bulk volume of a dosage form and/or to improve content uniformity of a dosage form.

Disintegrant may be highly/rapidly swellable, moderately swellable or slowly swellable such as vinylpyrrolidone polymers such as crospovidone, alginic acid, alginates, microcrystalline cellulose, cellulose and cellulose derivatives such as L-HPC (hydroxypropyl cellulose), polacrillin potassium, sodium starch glycolate, starch and starch derivatives such as pregelatinized starch, or carboxymethyl starch (e.g. Primogel® and Explotab®), resins, and like

Suitable anti-tacking agent is selected from the group consisting of, but is not limited to, stearates, stearic acid, vegetable oil, waxes, a blend of magnesium stearate and sodium lauryl sulfate, boric acid, surfactants, sodium benzoate, sodium acetate, sodium chloride, DL-Leucine, polyethylene glycol, sodium oleate, sodium lauryl sulfate, magnesium lauryl sulfate, talc, corn starch, amorphous silicon dioxide, syloid, metallic stearates, Vitamin E, Vitamin E TPGS, silica and combinations thereof.

Suitable binders include, but are not to be limited, cellulose derivatives include, but are not limited to be, methyl cellulose, hydroxypropyl methylcellulose, hydroxyethyl cellulose, hydroxypropyl cellulose, hydroxyethyl methylcellulose, ethylhydroxyethylcellulose, ethylmethylcellulose, hydroxymethylcellulose, hydroxymethylpropylcellulose, sodium carboxymethylcellulose, polyacrylamide derivatives, methacrylic acid derivatives, vinyl pyrrolidone polymers such as polyvinylpyrrolidone, starch derivative, polyalkylene oxide and copolymer thereof, alkylene oxide homopolymers, gums of plant, animal, mineral or synthetic origin, polyacrylic acid and copolymer thereof, polyvinyl alcohols, polyethylene glycol, poloxamer, and mixtures thereof. In some embodiments, the pharmaceutical formulation comprises a binder, such as lactose (e.g. spray-dried lactose, α-lactose, β-lactose, Tabletose®, various grades of Pharmatose®, Microtose® or Fast-FloC®), microcrystalline cellulose (various grades of Avicel®, Elcema®, Vivacel®, Ming Tai® or Solka-Floc®), hydroxypropylcellulose, L-hydroxypropylcellulose (low-substituted), hypromellose (HPMC) (e.g. Methocel E, F and K, Metolose SH of Shin-Etsu, Ltd, such as, e.g., the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, ethylcellulose, sodium carboxymethylcellulose, other cellulose derivatives, sucrose, dextrins, maltodextrins, starches or modified starches (including potato starch, maize starch and rice starch), calcium lactate, calcium carbonate, acacia, sodium alginate, agar, carrageenan, gelatin, guar gum, pectin, PEG, or povidone. In some embodiments the binder is povidone, such as povidone K 90.

Suitable glidants and lubricants may be incorporated such as stearic acid, magnesium stearate, calcium stearate or other metallic stearate, talc, waxes, and glycerides with high melting temperatures, colloidal silica, sodium stearyl fumarate, polyethyleneglycols, alkyl sulphates, light mineral oil, glyceryl behenate, hydrogenated vegetable oils, polyethylene glycols, or sodium benzoate.

Suitable solubility enhancers include, but not limited to, is selected from any or a combination of poly(ethylene glycol), including poly(ethylene glycol) having a molecular weight in the range of about 200 to about 5,000 Da, ethylene glycol, propylene glycol, non-ionic surfactants, tyloxapol, polysorbate 80, macrogol-15-hydroxystearate, phospholipids, lecithin, dimyristoyl phosphatidylcholine, dipalmitoyl phosphatidylcholine, distearoyl phosphatidylcholine, cyclodextrins,.alpha.-cyclodextrin,.beta.-cyclodextrin,.gamma.-cyclodextrin, hydroxyethyl-.beta.-cyclodextrin, hydroxypropyl-.beta.-cyclodextrin, hydroxyethyl-.gamma.-cyclodextrin, hydroxypropyl-.gamma.-cyclodexin, dihydroxypropyl-.beta.-cyclodextrin, Sulfobutylether-.beta.-cyclodextrin, sulfobutylether-.gamma.-cyclodextrin, glucosyl-.alpha.-cyclodextrin, glucosyl-.beta.-cyclodextrin, diglucosyl-.beta.-cyclodextrin, maltosyl-.alpha.-cyclodextrin, maltosyl-.beta.-cyclodextrin, maltosyl-.gamma.-cyclodextrin, maltotriosyl-.beta.-cyclodextrin, maltotriosyl-.gamma.-cyclodextrin, dimaltosyl-.beta.-cyclodextrin, methyl-.beta.-cyclodextrin, carboxyalkyl thioethers, hydroxypropyl methylcellulose, hydroxypropylcellulose, polyvinylpyrrolidone, vinyl acetate copolymers, vinyl pyrrolidone, sodium lauryl sulfate, dioctyl sodium sulfosuccinate and the likes. However, any or a combination of solubility enhancer(s), as known to or appreciated by a person skilled in the art, can be utilized to serve its intended purpose, as laid in the present disclosure, without departing from the scope and spirit of the present invention.

Suitable plasticizers include, but are not limited to, triacetin, triethyl acetate, acetylated monoglyceride, olive oil, acetyl tributyl citrate, acetyl triethyl citrate, glycerin, sorbitol, polyethylene glycol, polypropyleneglycol and like.

Stabilizer, such as antioxidants, can be used to inhibit or retard oxidative drug decomposition during storage of the pharmaceutical composition. In certain embodiments, the antioxidant or stabilizer includes, but are not limited to, ascorbic acid and its salts, α-tocopherol, sulfite salts, sodium sulfide, butylated hydroxyanisole, butylated hydroxytoluene, ascorbyl palmitate, propyl gallate, and the like.

Natural or synthetic sweeteners, in an amount of not more than 85 wt. % of total weight of the dosage form, include, but not limited to, sugar, mannitol, sorbitol, saccharose, saccharine, aspartame, acelsulphame K, or cyclamate.

Preferred sugars include dextrose, glucose, arabinose, ribose, arabinose, xylose, lyxose, xylol, allose, altrose, inositol, glucose, sorbitol, mannose, gulose, glycerol, idose, galactose, talose, trehalose, mannitol, erythritol, ribitol, xylitol, maltitol, isomalt, lactitol, sucrose, raffinose, maltose, fructose, lactose, dextrin, dextran, amylase and xylan.

Preferred coloring agents includes D&C dye/lake, FD&C dye, an FD&C lake, caramel, ferric oxide, a natural coloring agent, and a combination thereof. The amount of coloring agent used will vary as desired. Preferably the composition is non-toxic, edible, stable in light and air and free of potential hazards to human health.

Water soluble salts include sodium chloride, potassium chloride, calcium chloride or magnesium chloride, lithium chloride, lithium, sodium or potassium hydrogen phosphate, lithium, sodium or potassium dihydrogen phosphate, salts of organic acids such as sodium or potassium acetate, sodium bicarbonate, magnesium succinate, sodium benzoate, sodium citrate or sodium ascorbate.

Preferred acids include ascorbic acid, 2-benzene carboxylic acid, benzoic acid, fumaric acid, citric acid, maleic acid, serbacic acid, sorbic acid, edipic acid, edetic acid, glutamic acid, toluene sulfonic acid, water-soluble amino acids such as glycine, leucine, alanine, or methionine and tartaric acid; and like.

Osmotic agents includes sugar and salts as described above.

Surfactants and wetting agents include, but are not limited to, heptadecaethylene oxycetanol, lecithins, sorbitol monooleate, polyoxyethylene sorbitol monooleate, polyoxyethylene stearate, polyoxyethylen sorbitan monolaurate, benzalkonium chloride, nonoxynol 10, oxtoxynol 9, polysorbates for example 20, 40, 60 or 80, sorbitan mono-palmitate, sodium salts of fatty alcohol-sulfates such as sodium lauryl sulfate, sodium dodecylsulfate, sodium salts of sulfosuccinates such as sodium dioctylsulfosuccinate, partially esters of fatty acids with alcohols such as glycerine monostearate, partially esters of fatty acids with sorbitans such as sorbitan monolaurate, partially esters of fatty acids with polyhydroxyethylene sorbitans such as polyethyleneglycol sorbitan monolaurate, -monostearate or -monooleate, ethers of fatty alcohols with polyhydroxyethylene, esters of fatty acids with polyhydroxyethylene, copolymers of ethylenoxide and propylenoxide (Pluronic®) and ethoxylated triglycerides.

Various useful preservatives include, but are not limited to, parabens such as methylparaben, propylparaben, butyl paraben and their salts, sorbic acid, sodium sorbate, potassium sorbate, calcium sorbate, benzoic acid, sodium benzoate, potassium benzoate, calcium benzoate, methyl hydroxybenzoate, ethyl para-hydroxybenzoate, sodium ethyl para-hydroxybenzoate, sodium metabisulphite, chlorhexidine, diazolidinyl urea, sodium citrate, butylated hydroxyl toluene (BHT), butylated hydroxyl anisole (BHA), tocopherol, ethylenediamine tetraacetic acid, propyl gallate, quaternary compounds, e.g. benzalkonium chloride and cetylpyridinium chloride, phenyl ethyl alcohol and combinations thereof. The preservative is present in an amount of about 0.001% w/w to about 4% w/w of the pharmaceutical composition, particularly in an amount of about 0.001% w/w to about 3% w/w of the pharmaceutical composition.

In some embodiments, the pharmaceutical formulation comprises one or more excipients selected from crystallization retarders, such as Povidone, etc.; solubilizing agents (also known as surfactants), such as anionic surfactants (e.g. Pluronic or Povidone), cationic surfactants, nonionic surfactants, and/or zwitterionic surfactants; colouring agents, including dyes and pigments, such as Iron Oxide Red or Yellow, titanium dioxide, and/or talc; and/or pH control agents, such as citric acid, tartaric acid, fumaric acid, sodium citrate, dibasic calcium phosphate, and/or dibasic sodium phosphate.

The suspending agents or viscosity enhancing agent are selected from the group comprising cellulose derivatives such as hydroxyethyl cellulose, hydroxypropyl cellulose, hydroxypropylmethyl cellulose, methylcellulose, carboxymethyl cellulose and its salts/derivatives e.g., carboxymethyl cellulose sodium, microcrystalline cellulose, and co-processed spray dried forms of microcrystalline cellulose and carboxymethyl cellulose sodium (such as Avicel® RC-501, Avicel® RC-581, Avicel® RC-591, and Avicel® CL-611); xanthan gum; carbomers (such as those available under the trade name Carbopol); gums such as locust bean gum, tragacanth gum, arabinogalactan gum, agar gum, gellan gum, guar gum, apricot gum, karaya gum, sterculia gum, acacia gum, gum arabic, and carrageenan; pectin; propylene glycol alginate, dextran; gelatin; polyethylene glycols; polyvinyl compounds such as polyvinyl acetate, polyvinyl alcohol, and polyvinyl pyrrolidone; sugar alcohols such as xylitol and mannitol; colloidal silica; maltodextrin, starch; and mixtures thereof. The suspending agents are present in an amount of about 0.01% to about 20% w/w of the pharmaceutical composition.

Carrier/vehicle/solvent includes aqueous, non-aqueous, or combination thereof in any proportion such as, but are not limited to, water, alcohol, polyethylene glycol, propylene glycol or glycerin, buffers, oil, or combinations thereof. Oils include peanut oil, soybean oil, corn oil, olive oil, sesame oil, cotton seed oil, acetylated glycerides, ethyl oleate, mineral oil, fatty acid esters, mono- or di-fatty acid esters of polyethylene glycols, or glyceryl mono-oleate. By “aqueous carrier” is meant a carrier comprising of water, or a combination of water and a water-miscible organic solvent or solvents. Water-miscible solvents include but are not limited to propylene glycol, polyethylene glycol and alcohol such as ethanol. By “non-aqueous carrier” is meant the carrier does not include water. The carrier can also include one more pharmaceutically acceptable excipients which can be in dissolved or dispersed form. The carrier is present in an amount from about 2.5% w/w to about 99% w/w, about 10% w/w to about 99% w/w, particularly from about 30% w/w to about 95% w/w, particularly from about 40% w/w to about 95% w/w, particularly from about 50% w/w to about 95% w/w and particularly from about 60% w/w to about 95% w/w of the total weight of the pharmaceutical composition.

The anticaking agent helps to improve the re-suspendability of the liquid pharmaceutical formulation. Various useful anticaking agents include, but are not limited to, colloidal silica and/or colloidal silicon dioxide, calcium phosphate tribasic, magnesium oxide, magnesium silicate, calcium silicate, and combinations thereof. The anticaking agents are present in an amount of about 0.1% to about 10% w/w of the total weight of the pharmaceutical composition.

The flavoring agents, include, but are not limited to, flavors such as banana, lemon, orange, grape, lime and grapefruit, vanilla, bubble gum, peppermint, fantasy fruit masking flavor (as commercially available), and fruit essence, including apple, banana, pear, peach, strawberry, raspberry, cherry, plum, pineapple, apricot; synthetic flavor oils and flavoring aromatics and/or natural oils, extracts from plant leaves, flowers, fruits such as cinnamon oil, oil of wintergreen, peppermint oils, clove oil, citrus oil, bay oil, anise oil, eucalyptus, thyme oil, cedar leaf oil, oil of nutmeg, oil of sage, oil of bitter almonds, and cassia oil; maltol, ethyl vanillin, menthol, citric acid, fumaric acid, ethyl maltol, and tartaric acid and combinations thereof. The flavoring agents are present in an amount of about 0.01% to about 7% w/w of the total weight of the pharmaceutical composition.

Functionality of pharmaceutically acceptable additive and tentative range for concentration to be used in the pharmaceutical dosage form according to the functionality also described in Handbook of Pharmaceutical Excipients, 6th Edition, Edited by Raymond C Rowe, Paul J Sheskey and Marian E Quinn.

The above-listed ingredients are for information only, which does not limit the scope of the present invention. Any ingredient used in the pharmaceutical composition that fulfills the subject matter of the present invention is within the scope of the present invention. Therefore, any or a combination of one or more excipient(s), as known to or appreciated by a person skilled in the art, can be utilized to serve its intended purpose, as laid in the present disclosure, without departing from the scope and spirit of the present invention.

In some embodiments, one ingredient or agent may also act for multiple functions.

The presently disclosed pharmaceutical composition can be packaged in a suitable pack/container such as amber colored polyethylene terephthalate (PET) bottle, glass bottle, high-density polyethylene (HDPE) bottle, low-density polyethylene (LDPE) bottle, polypropylene (PP) bottle, packets, pouches, sachets, blister, strip and the like.

In certain embodiments, the pharmaceutical formulation according to any one of the preceding embodiments may be formulated as any solid oral dosage form or a liquid oral dosage form. In certain embodiments, the oral pharmaceutical dosage form is selected from any or a combination of tablets (monolayer or multilayers coated or uncoated tablets), capsules (soft gelatin capsules, hard gelatin capsules, HPMC capsules, or HPMCP capsules), a capsule-in-capsule, tablet-in-capsule, lozenges, troches, ovules, solutions, emulsions, suspensions, syrups, elixirs, powders and granules for reconstitution, dispersible powders and granules, medicated gums, chewing tablets, effervescent tablets, multi-particulate dosage forms (multi-particulate filled in capsule, or compressed with one or more pharmaceutical additives to form tablet), and the likes. The term “multi-particulate” as used herein means a plurality of discrete particles, pellets, mini-tablets, mini-capsules, or combinations thereof.

In certain embodiments, the pharmaceutical formulation according to any one of the preceding embodiments includes at least one region comprising a mixture of at least one of the drugs, the acid neutralizer, the alkaline salt of an oral absorption promoter, and the at least one intestinal enzyme inhibitor.

In certain embodiments, the pharmaceutical formulation according to any one of the preceding embodiments includes at least two regions that are partially (attached to each other at some extent) or completely separated from each other, wherein the drug is present in a first region, and at least one of the acid neutralizer, the alkaline salt of an oral absorption promoter, and the intestinal enzyme inhibitor is present in a second region. In certain embodiments, the first region optionally includes at least one of the acid neutralizer, the alkaline salt of an oral absorption promoter, and the intestinal enzyme inhibitor.

In certain embodiments, the pharmaceutical formulation according to any one of the preceding embodiments includes at least three regions that are partially (attached to each other at some extent) or completely separated from each other, wherein the drug is present in a first region and at least one of the acid neutralizer, and the alkaline salt of an oral absorption promoter is present in a second region, and the at intestinal enzyme inhibitor is present in a third region. In certain embodiments, the first region optionally includes at least one of the acid neutralizer, the alkaline salt of an oral absorption promoter, and the intestinal enzyme inhibitor.

In certain embodiments, the pharmaceutical formulation according to any one of the preceding embodiments includes a plurality of regions that are partially (attached to each other at some extent) or completely separated from each other, wherein each region include at least one of the drug, the acid neutralizer, the alkaline salt of an oral absorption promoter, and the intestinal enzyme inhibitor.

In certain embodiments, the pharmaceutical formulation according to any one of the preceding embodiments is formulated as a monolayer or a multilayer, coated or uncoated tablet. The presently disclosed subject matter does not limit to a sequence of addition of any ingredient(s), such as the drug, the alkaline salt of an oral absorption promoter, the acid neutralizer, and the intestinal enzyme inhibitor during fabricating the monolayer or multilayer, coated or uncoated tablet. For example, at least one of the drug, the acid neutralizer, the alkaline salt of an oral absorption promoter, and the intestinal enzyme inhibitor may be contained in (a) a same or different layer of coated or uncoated tablet; (b) one or more coating layers surrounding the monolayer or multilayer coated tablet; (c) any layer of the monolayer or multilayer tablet and one or more coating layers surrounding the monolayer or multilayer tablet; or (d) combination thereof.

In certain embodiments, the pharmaceutical formulation according to any one of the preceding embodiments is formulated as a coated or uncoated capsule. The presently disclosed subject matter does not limit to a sequence of addition of any ingredient(s), such as the drug, the alkaline salt of an oral absorption promoter, the acid neutralizer, and the intestinal enzyme inhibitor during fabricating the coated or uncoated capsule. For example, at least one of the drug, the alkaline salt of an oral absorption promoter, the acid neutralizer, and the intestinal enzyme inhibitor may be included, as an individual ingredient and/or a physical mixture of two or more ingredients (a) in coated or uncoated capsule, (b) in capsule and one or more coating layers surrounding the filled capsule; (c) in inner capsule and outer capsule (for capsule-in-capsule type of dosage form), or (d) in inner capsule and outer capsule (for capsule-in-capsule type of dosage form), and one or more coating layers surrounding the filled capsule-in-capsule.

In certain embodiments, the pharmaceutical formulation according to any one of the preceding embodiments is formulated as a multicompartment dosage form, such as through coated monolayer tablet, coated capsule, coated or uncoated multilayer tablet, coated or uncoated tablet-in-tablet, coated or uncoated tablet-in-capsule, coated or uncoated capsule-in-capsule, or any novel multicompartment dosage form, wherein the drug is present in one compartment, and any or any combination of the alkaline salt of oral absorption promoter, the acid neutralizer, and the intestinal enzyme inhibitor is present in other, same or different, compartment of the multicompartment dosage form. The sole purpose of the multicompartment dosage form is to improve the stability of the pharmaceutical formulation by minimizing direct contact of the drug with at least one of the alkaline salt of an oral absorption promoter, the acid neutralizer, and the intestinal enzyme inhibitor.

In certain embodiments, the pharmaceutical formulation according to any one of the preceding embodiments is formulated as multiparticulates. The presently disclosed subject matter does not limit to a sequence of addition of any ingredient(s), such as the drug, the alkaline salt of an oral absorption promoter, the acid neutralizer, and the intestinal enzyme inhibitor while fabricating the coated or uncoated multiparticulates. For example, at least one of the drug, the alkaline salt of an oral absorption promoter, the acid neutralizer, and the intestinal enzyme inhibitor may be included in the same or different populations of particulates (such as granular form, pellets, beads, minitablets, and mini-capsule) making up the multiparticulates, or may be contained in a single or multiple coating layer surrounding a seed core (such as a non-pareil seed, such as sugar sphere, microcrystalline cellulose sphere, silicon seed, etc.) or any combination thereof. Another method for manufacturing the multiparticulate cores of this embodiment is the extrusion and spheronization process. In certain embodiments, if the dosage form is multiparticulates, capsules such as hard or soft gelatin capsules can suitably be used to contain the multiparticulate. In certain embodiments, the multiparticulates can be compressed, optionally with other additives, into tablet form. In certain embodiments, for ease of administration, the multiparticulates dosage form can be sprinkled on any liquid medium or can be reconstituted using suitable amount of any liquid medium prior to administration.

In certain embodiments, the pharmaceutical formulation according to any one of the preceding embodiments is formulated as the liquid oral dosage form. In certain embodiments, the oral liquid dosage form, in addition to the drug and at least one of the alkaline salt of an oral absorption promoter, the acid neutralizer, and the intestinal enzyme inhibitor, may also include any or a combination of one or more excipients such as, but not limited to, sweetening, flavoring agents, preservatives, anti-oxidant, coloring matter or dyes, emulsifying and/or suspending agents and diluents such as water, ethanol, propylene glycol, and glycerin. In certain embodiment, the liquid oral dosage form is complete or partially in a liquid form. In certain embodiments, the complete or partially liquid form composition is substantially a water free composition. The term “substantially water free composition” refers to a liquid composition that contains less than about 50% (v/v) of water, preferably less than about 30% (v/v) of water, more preferably less than about 10% (v/v) of water, even more preferably less than about 5% (v/v) of water, even more preferably less than about 0.5% (v/v) of water, yet even more preferably less than about 0.1% (v/v) of water, and is still more preferably free of water. In certain embodiments, the solid oral dosage form, such as powders and granules for reconstitution, can be prepared as the liquid dosage form shortly before administration. In this case, the composition should preferably be prepared shortly before administration to the subject/patient, such as the powder or granules of the composition mixed with water just prior to administration by the patient in need thereof, and in some cases, prolonged storage periods should be avoided. In certain embodiments, the pharmaceutical formulation is formulated as a capsule dosage form, wherein the capsule dosage form is hard gelatin capsule, soft gelatin capsule, capsule-in-capsule or tablet-in-capsule, and wherein the capsule dosage form is filled with the pharmaceutical formulation, complete or partially, in a liquid form.

In certain embodiments, the intestinal enzyme inhibitor of the pharmaceutical formulation is physically separated, within the dosage form, from at least one of the drug, the alkaline salt of an oral absorption promoter, and the acid neutralizer through the enteric coat, wherein the intestinal enzyme inhibitor is in a particulate or multiparticulates form, wherein the particulate or multiparticulates is coated with one or more enteric coating materials which are soluble/degradable at pH of greater than about 5.5 in the intestinal region, thereby allowing the release of a substantial amount of the intestinal enzyme inhibitor in the intestinal region, more specifically in the upper part of the intestinal region.

However, the presently disclosed subject matter is not limited to the art of fabricating the pharmaceutical formulation. Any other dosage form, as known to or appreciated by a person skilled in the art, also including the dosage form that minimizes direct contact of the drug or acid neutralizer with at least one of the alkaline salt of an oral absorption promoter, and the intestinal enzyme inhibitor, can be utilized to serve for its intended purpose, as laid in the present disclosure, without departing from the scope and spirit of the present invention.

In certain embodiments, the enteric coating material includes, but is not limited to, at least one polymer selected from the group consisting of a copolymer of methacrylic acid and methyl methacrylate (1:1), a copolymer of methacrylic acid and methyl methacrylate (1:2), a copolymer of methacrylic acid and ethyl acrylate (1:1), hydroxypropyl methylcellulose acetate succinate, hydroxypropyl methylcellulose phthalate, cellulose acetate phthalate, crosslinked polyacrylic polymers, and polyvinyl acetate phthalate. In certain embodiments, the enteric coating material includes, but are not limited to, acrylic acid derivatives such as eudragit S 100, eudragit FS, eudragit L 100, eudragit L 100-55, or mixtures thereof, cellulose esters such as cellulose acetate phthalate, cellulose acetate trimellitate, hydroxypropylmethyl cellulose acetate succinate, hydroxypropylmethyl cellulose phthalate, polyvinyl derivatives such as polyvinyl acetate phthalate, polysaccharide, shellac, zein, pectin, amylase starch and starch derivatives, and like. Other than what is mentioned in the presently disclosed subject matter, the enteric coating material also includes any ingredient, material, or polymer that is soluble at pH above about 5.5 (including degrade or digest by only intestine enzymes) but insoluble, or alternatively swells or gels, at pH below about 5.5.

In some embodiments, the enteric coating material is present in an amount of about 0.1% w/w to about 55% w/w; about 0.2% w/w to about 35% w/w; about 0.4% w/w to about 15% w/w; or about 0.5% w/w to about 5% w/w of the pharmaceutical composition. In certain embodiments, the enteric coating material is present in an amount of less than 55%, less than 35%, less than 15%, less than 12%, less than 10%, less than 8%, less than 7%, less than 6%, or less than 5% w/w of the total weight of the pharmaceutical composition.

In this specification, a number of documents including patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The present invention also relates to a pharmaceutical composition of the invention for use as a medicament. In one embodiment, the pharmaceutical composition is administered orally, and may be used for any of the following medical treatments, but not limited to, all preferably relating one way or the other to diabetes: (i) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C; (ii) delaying or preventing diabetic disease progression, such as progression in type 2 diabetes, delaying the progression of impaired glucose tolerance (IGT) to insulin requiring type 2 diabetes, and/or delaying the progression of non-insulin requiring type 2 diabetes to insulin requiring type 2 diabetes; (iii) improving β-cell function, such as decreasing β-cell apoptosis, increasing B-cell function and/or B-cell mass, and/or for restoring glucose sensitivity to β-cells; (iv) prevention and/or treatment of cognitive disorders; (v) prevention and/or treatment of eating disorders, such as obesity, e.g. by decreasing food intake, reducing body weight, suppressing appetite, inducing satiety; treating or preventing binge eating disorder, bulimia nervosa, and/or obesity induced by administration of an antipsychotic or a steroid; reduction of gastric motility; and/or delaying gastric emptying; (vi) prevention and/or treatment of diabetic complications, such as neuropathy, including peripheral neuropathy; nephropathy; or retinopathy; (vii) improving lipid parameters, such as prevention and/or treatment of dyslipidemia, lowering total serum lipids; lowering HDL; lowering small, dense LDL; lowering VLDL: lowering triglycerides; lowering cholesterol; increasing HDL; lowering plasma levels of lipoprotein a (Lp(a)) in a human; inhibiting generation of apolipoprotein a (apo(a)) in vitro and/or in vivo; (iix) prevention and/or treatment of cardiovascular diseases, such as syndrome X; atherosclerosis; myocardial infarction; coronary heart disease; stroke, cerebral ischemia; an early cardiac or early cardiovascular disease, such as left ventricular hypertrophy; coronary artery disease; essential hypertension; acute hypertensive emergency; cardiomyopathy; heart insufficiency; exercise tolerance; chronic heart failure; arrhythmia; cardiac dysrhythmia; syncopy; atheroschlerosis; mild chronic heart failure; angina pectoris; cardiac bypass reocclusion; intermittent claudication (atheroschlerosisoblitterens); diastolic dysfunction; and/or systolic dysfunction; (ix) prevention and/or treatment of gastrointestinal diseases, such as inflammatory bowel syndrome; small bowel syndrome, or Crohn's disease; dyspepsia; and/or gastric ulcers; (x) prevention and/or treatment of critical illness, such as treatment of a critically ill patient, a critical illness poly-nephropathy (CIPNP) patient, and/or a potential CIPNP patient; prevention of critical illness or development of CIPNP; prevention, treatment and/or cure of systemic inflammatory response syndrome (SIRS) in a patient; and/or for the prevention or reduction of the likelihood of a patient suffering from bacteraemia, septicaemia, and/or septic shock during hospitalisation; and/or (xi) prevention and/or treatment of polycystic ovary syndrome (PCOS).

In some embodiments, the invention relates to a pharmaceutical formulation according to any one of the preceding embodiments is recommended to be administered in a fasted state of stomach.

In some embodiments, the invention relates to a pharmaceutical formulation according to any one of the preceding embodiments is recommended to be administered with 120 mL, 125 mL or 240 mL of drinking water.

### EXAMPLES

The presently disclosed subject matter will now be illustrated by the following examples which are not to be taken as limiting.

### Example 1: Determination of % Assay of SNAC in Absence of Alkalizer

100 mg of Salcaprozate Sodium (SNAC) was added in the following media. pH of the media or the resultant mixture (before and after addition of the SNAC) and % assay of the SNAC was determined after 5 minutes of mixing.

TABLE 1
% assay of SNAC in absence of alkalizer

| | pH of media or | pH of mixture | | |
|---|---|---|---|---|
| | mixture before | after addition | | |
| Media | addition of SNAC | of SNAC | % Assay of SNAC | Appearance |
| Added 100 mg of | 1.14 | 1.18 | 0.013 | White |
| SNAC in 35 mL | | | | Precipitates |
| of 0.1 N HCl | | | | |
| Added 100 mg of | 1.67 | 1.68 | 0.663 | White |
| SNAC in the | | | | Precipitates |
| mixture of 35 mL | | | | |
| of 0.1 N HCl + | | | | |
| 125 mL of water | | | | |

SNAC has been determined to be insoluble, exhibiting white precipitates. This finding is further confirmed by the % assay, which is noted to be less than 1%. SNAC did not increase the pH of the resultant media.

### Example 2: Determination of % Assay of SNAC in Presence of Alkalizer: SNAC Added Prior to the Addition of Alkalizer

100 mg of Salcaprozate Sodium (SNAC) was added prior to the addition of alkalizer (sodium bicarbonate) in the following media. pH of the media or the resultant mixture (before and after addition of the SNAC) and % assay of the SNAC was determined after 5 minutes of mixing.

TABLE 2
% assay of SNAC: SNAC added prior to the addition of alkalizer

| | pH of media or | | | |
|---|---|---|---|---|
| | mixture before | pH of the resultant | | |
| | addition of | mixture after | % Assay of | |
| Media | SNAC | addition of SNAC | SNAC | Appearance |
| Added 100 mg of SNAC in | 1.67 | 1.68 | 0.663 | White |
| the mixture of 35 mL of | | | | Precipitates |
| 0.1 N HCl + 125 mL of | | | | |
| water | | | | |
| Added 100 mg of SNAC in | 1.67 | 1.84 | 0.118 | White |
| the mixture of 35 mL of | | | | Precipitates |
| 0.1 N HCl + 125 mL of | | | | |
| water, then added 100 mg | | | | |
| of Sodium Bicarbonate | | | | |
| Added 100 mg of SNAC in | 1.67 | 5.19 | 3.917 | White |
| the mixture of 35 mL of | | | | Precipitates |
| 0.1 N HCl + 125 mL of | | | | |
| water, then added 300 mg | | | | |
| of Sodium Bicarbonate | | | | |

Although the SNAC is still exhibiting white precipitates, its solubility or dissolution is increasing from 0.663% (without sodium bicarbonate) to 3.917% after addition of 300 mg of Sodium Bicarbonate, however, it was surprisingly found that 100 mg of Sodium Bicarbonate is not sufficient to improve the dissolution or release property of SNAC.

### Example 3: Determination of % Assay of SNAC in Presence of Alkalizer: SNAC Added after the Addition of Alkalizer

100 mg of Salcaprozate Sodium (SNAC) was added after the addition of alkalizer (sodium bicarbonate) in the following media. pH of the media or the resultant mixture (before and after addition of the SNAC) and % assay of the SNAC was determined after 5 minutes of mixing.

TABLE 3
% assay of SNAC: SNAC added after the addition of alkalizer

| | pH of media or | pH of the resultant | | |
|---|---|---|---|---|
| | mixture before | mixture after | | |
| | addition of | addition of | % Assay of | |
| Media | SNAC | SNAC | SNAC | Appearance |
| Added 100 mg of SNAC in | 1.14 | 1.18 | 0.013 | White |
| 35 mL of 0.1 N HCl | | | | Precipitates |
| Added 300 mg of Sodium | 5.08 | 5.88 | 14.824 | White |
| Bicarbonate in 35 mL of | | | | Precipitates |
| 0.1 N HCl, then added | | | | |
| 100 mg of SNAC | | | | |
| Added 300 mg of Sodium | 5.08 | 6.01 | 23.457 | White |
| Bicarbonate in 35 mL of | | | | Precipitates |
| 0.1 N HCl, then added | | | | |
| 100 mg of SNAC, then | | | | |
| added 125 mL of Water | | | | |

When 300 mg of Sodium Bicarbonate dissolved or released in the 35 mL of 0.1N HCl prior to the addition of 100 mg of SNAC, it surprisingly improves the solubility or dissolution of SNAC at greater extent from 0.013% to 14.824% (in absence of 125 mL of water-mimic situation where subject administer a pharmaceutical formulation without water) or 23.457% (after addition of 125 ml of water-mimic situation where subject administer the pharmaceutical formulation with 125 mL of water).

### Example 4: Determination of % Assay of SNAC in Presence of Alkalizer: SNAC Added after the Addition of Alkalizer

100 mg of Salcaprozate Sodium (SNAC) was added after the addition of alkalizer (sodium bicarbonate) in the following media. pH of the media or the resultant mixture (before and after addition of the SNAC) and % assay of the SNAC was determined after 5 minutes of mixing.

TABLE 4
% assay of SNAC: SNAC added after the addition of alkalizer

| | pH of media or | pH of the | % | |
|---|---|---|---|---|
| | mixture before | resultant mixture | Assay | |
| | addition of | after addition of | of | |
| Media | SNAC | SNAC | SNAC | Appearance |
| Added 100 mg of SNAC in | 1.67 | 1.68 | 0.663 | White |
| the mixture of 35 mL of | | | | Precipitates |
| 0.1 N HCl + 125 mL of | | | | |
| water | | | | |
| (No Sodium Bicarbonate) | | | | |
| Added 100 mg of SNAC in | 1.67 | 5.19 | 3.917 | White |
| the mixture of 35 mL of | | | | Precipitates |
| 0.1 N HCl + 125 mL of | | | | |
| water, then added 300 mg | | | | |
| of Sodium Bicarbonate | | | | |
| (Sodium Bicarbonate | | | | |
| added after SNAC) | | | | |
| Added 300 mg of Sodium | 5.10 | 5.31 | 79.079 | Clear |
| Bicarbonate in the mixture | | | | Solution |
| of 35 mL of 0.1 N HCl + | | | | |
| 125 mL of water, then | | | | |
| added 100 mg of SNAC | | | | |
| (Sodium Bicarbonate | | | | |
| added prior to SNAC) | | | | |

Above result further confirmed that release or dissolution of sodium bicarbonate (alkalizer) prior to addition or release of the SNAC greatly improves the solubility or dissolution of SNAC at greater extent from 0.663% (without sodium bicarbonate) or 3.917% (addition or release of sodium bicarbonate after the SNAC) to 79.079%, which makes the resultant solution clear.

### Example 5: Determination of % Assay of SNAC in Presence of Alkalizer: Alkalizer Added Prior to, Concurrently with, or after the Addition of SNAC

100 mg of Salcaprozate Sodium (SNAC) was added after, concurrently with, or prior to the addition of alkalizer (sodium bicarbonate) in the following media. pH of the media or the resultant mixture (before and after addition of the SNAC) and % assay of the SNAC was determined after 5 minutes of mixing.

TABLE 5
% assay of SNAC: SNAC added prior to, concurrently with, or after the addition
of alkalizer

| | pH of media or | pH of the | % | |
|---|---|---|---|---|
| | mixture before | resultant mixture | Assay | |
| | addition of | after addition of | of | |
| Media | SNAC | SNAC | SNAC | Appearance |
| Added 100 mg of SNAC in | 1.67 | 1.68 | 0.663 | White |
| the mixture of 35 mL of | | | | Precipitates |
| 0.1 N HCl + 125 mL of | | | | |
| water | | | | |
| (No Sodium Bicarbonate) | | | | |
| Added 100 mg of SNAC in | 1.67 | 5.19 | 3.917 | White |
| the mixture of 35 mL of | | | | Precipitates |
| 0.1 N HCl + 125 mL of | | | | |
| water, then added 300 mg | | | | |
| of Sodium Bicarbonate | | | | |
| (Sodium Bicarbonate | | | | |
| added after SNAC) | | | | |
| Added the mixture of | 1.67 | 5.37 | 10.287 | White |
| 300 mg Sodium | | | | Precipitates |
| Bicarbonate and 100 mg | | | | |
| SNAC in the mixture of | | | | |
| 35 mL of 0.1 N HCl + | | | | |
| 125 mL of water | | | | |
| (Sodium Bicarbonate | | | | |
| added concurrently with | | | | |
| SNAC) | | | | |
| Added 300 mg of Sodium | 5.10 | 5.31 | 79.079 | Clear |
| Bicarbonate in the mixture | | | | Solution |
| of 35 mL of 0.1 N HCl + | | | | |
| 125 mL of water, then | | | | |
| added 100 mg of SNAC | | | | |
| (Sodium Bicarbonate | | | | |
| added prior to SNAC) | | | | |

Present inventor surprisingly found that the sequence of release or addition of sodium bicarbonate (alkalizer) with respect to the release or addition of SNAC impacted the solubility or dissolution property of SNAC to a greater extent, for example,
release or dissolution of sodium bicarbonate (alkalizer) after addition or release of SNAC into media improved dissolution of the SNAC from 0.663% (addition or release of sodium bicarbonate after the SNAC) to 3.917%, making solution with white precipitates.
release or dissolution of sodium bicarbonate (alkalizer) concurrently with addition or release of SNAC into media improved dissolution of the SNAC from 0.663% (addition or release of sodium bicarbonate after the SNAC) to 10.287%, making solution with white precipitates.
release or dissolution of sodium bicarbonate (alkalizer) prior to addition or release of SNAC into media greatly improved dissolution of the SNAC from 0.663% (addition or release of sodium bicarbonate after the SNAC) to 79.079%, making clear solution.

### Example 6: Determination of % Release of SNAC: Alkalizer Release Prior to or Concurrently with SNAC

% release of 100 mg SNAC and 300 mg SNAC was determined in 160 mL of acidic medium having pH of less than 2 over 45 minutes using USP apparatus 2 (modified small paddle), 50 RPM, at 37° C.

TABLE 6
% release of SNAC: Alkalizer release prior to or concurrently with SNAC

| | % Release of SNAC | | |
|---|---|---|---|
| | 300 mg of Sodium | 300 mg of Sodium | 400 mg of Sodium |
| | Bicarbonate released | Bicarbonate released | Bicarbonate released |
| Time | concurrently with the | prior to the release of | prior to the release of |
| (Minutes) | release of 100 mg of SNAC | 100 mg of SNAC | 300 mg of SNAC |
| 5 | 3.64 | 72.94 | 88.70 |
| 10 | 8.89 | 54.68 | 84.19 |
| 20 | 10.55 | 39.19 | 68.68 |
| 30 | 11.94 | 33.88 | 54.44 |
| 50 | 13.71 | 29.60 | 50.00 |
| Appearance | White Precipitates | Clear solution at very | Clear solution at very |
| | | beginning, later white | beginning, later white |
| | | precipitates observed | precipitates observed |

Above dissolution data further confirmed that sequence or release of alkalizer (sodium bicarbonate) from the pharmaceutical formulation greatly impacted the % release or dissolution of SNAC.

### Example 7: Determination of % Release of SNAC: Alkalizer (70 mg of Magnesium Oxide) Release Prior to or Concurrently with SNAC

% release of 300 mg SNAC was determined in 160 mL of acidic medium having pH of less than 2 over 45 minutes using USP apparatus 2 (modified small paddle), 50 RPM, at 37° C.

TABLE 7
% release of SNAC: Alkalizer release prior to or concurrently with SNAC

| | 70 mg of magnesium oxide | | 70 mg of magnesium oxide | |
|---|---|---|---|---|
| | released concurrently | | released prior | |
| | with the release of 300 mg of SNAC | | to the release of 300 mg of SNAC | |
| Time | % Release of | | % Release | pH of |
| (Minutes) | SNAC | pH of Media | of SNAC | Media |
| 2 | 1.00 | 2.45 | 97.0 | 6.47 |
| 5 | 6.7 | 5.55 | 97.0 | |
| 10 | 16.4 | 6.14 | 97.2 | |
| 15 | 22.8 | 6.32 | 97.5 | |
| 30 | 35.6 | 6.57 | 97.6 | |
| 45 | 45.8 | 6.69 | 97.4 | |

| | | |
|---|---|---|
| Appearance | White Precipitates | Clear Solution |

Even though attaining similar or better pH of the resultant media, above dissolution data further confirmed that sequence or release of alkalizer (sodium bicarbonate) from the pharmaceutical formulation greatly impacted the % release or dissolution of SNAC. Further, selection of appropriate alkalizer is also crucial to stabilize the solubility or % dissolved content of SNAC in the resultant media.

### Example 8

Various pharmaceutical formulations according to the presently disclosed subject matter could be manufactured using the formulation composition as listed below.

TABLE 8
Capsule Composition for Semaglutide

| Sr. No. | Ingredient Name | Mg/Tablet | % w/w |
|---|---|---|---|
| 1 | Semaglutide | 10.00 | 1.63 |
| 2 | Salcaprozate Sodium | 250.00 | 40.65 |
| 3 | Sodium Bicarbonate | 300.00 | 48.78 |
| 4 | Microcrystalline Cellulose | 23.50 | 3.82 |
| 5 | Polyvinyl Pyrrolidone | 25.00 | 4.07 |
| 6 | Magnesium Stearate | 6.50 | 1.06 |

| | | |
|---|---|---|
| Total Weight | 615.00 | 100.00 |

TABLE 9
Multi-Layer Semaglutide Tablet Composition

| | | | |
|---|---|---|---|
| Sr. No. | Ingredient Name | Mg/Tablet | % w/w |

First Layer

| | | | |
|---|---|---|---|
| 1 | Semaglutide | 10.00 | 1.67 |
| 2 | Microcrystalline Cellulose | 59.00 | 9.83 |
| 3 | Polyvinyl Pyrrolidone | 5.25 | 0.88 |
| 4 | Magnesium Stearate | 0.75 | 0.12 |

Second Layer

| | | | |
|---|---|---|---|
| 5 | Salcaprozate Sodium | 200.00 | 33.33 |
| 6 | Sodium Bicarbonate | 250.00 | 41.67 |
| 7 | Microcrystalline Cellulose | 44.75 | 7.46 |
| 8 | Polyvinyl Pyrrolidone | 25.00 | 4.17 |
| 9 | Magnesium Stearate | 5.25 | 0.87 |

| | | |
|---|---|---|
| Total Weight | 600.00 | 100.00 |

TABLE 10
Multi-Layer Semaglutide Tablet Composition

| | | | |
|---|---|---|---|
| Sr. No. | Ingredient Name | Mg/Tablet | % w/w |

First Layer

| | | | |
|---|---|---|---|
| 1 | Semaglutide | 10.00 | 1.18 |
| 2 | Microcrystalline Cellulose | 59.00 | 6.94 |
| 3 | Salcaprozate Sodium | 200.00 | 23.52 |
| 4 | Polyvinyl Pyrrolidone | 23.05 | 2.71 |
| 5 | Magnesium Stearate | 2.95 | 0.35 |

Second Layer

| | | | |
|---|---|---|---|
| 6 | Sodium Bicarbonate | 250.00 | 29.41 |
| 7 | Sodium Starch Glycolate | 30.00 | 3.53 |
| 8 | Microcrystalline Cellulose | 244.45 | 28.75 |
| 9 | Polyvinyl Pyrrolidone | 25.00 | 2.94 |
| 7 | Magnesium Stearate | 5.55 | 0.65 |

| | | |
|---|---|---|
| Total Weight | 850.00 | 100.00 |

TABLE 11
Single Layer Tablet Composition or Capsule Composition
for Semaglutide

| Sr. No. | Ingredient Name | Mg/Tablet | % w/w |
|---|---|---|---|
| 1 | Semaglutide | 25.00 | 5.3 |
| 2 | Salcaprozate Sodium | 300.00 | 63.2 |
| 3 | Magnesium Oxide | 70.00 | 14.7 |
| 4 | Microcrystalline Cellulose | 60.00 | 12.6 |
| 5 | Polyvinyl Pyrrolidone | 15.00 | 3.2 |
| 6 | Magnesium Stearate | 5.00 | 1.1 |

| | | |
|---|---|---|
| Total Weight | 475.00 | 100.00 |

TABLE 12
Multi-Layer Semaglutide Tablet Composition

| | | | | |
|---|---|---|---|---|
| Sr. | | | Mg/ | % |
| No. | Ingredient Name | Function | Tablet | w/w |

First Layer

| | | | | |
|---|---|---|---|---|
| 1 | Semaglutide | Drug | 10.00 | 1.67 |
| 2 | Microcrystalline | Diluent | 59.00 | 9.83 |
| | Cellulose | | | |
| 3 | Salcaprozate Sodium | Oral absorption promoter | 200.00 | 33.33 |
| 4 | Polyvinyl Pyrrolidone | Binder | 5.25 | 0.88 |
| 5 | Magnesium Stearate | Lubricant | 0.75 | 0.13 |

Second Layer

| | | | | |
|---|---|---|---|---|
| 6 | Magnesium Oxide | Acid Neutralizing Agent | 70.00 | 11.67 |
| 7 | Microcrystalline | Diluent | 194.72 | 32.45 |
| | Cellulose | | | |
| 8 | Manganese Gluconate | Metal salt | 0.03 | 0.01 |
| 9 | Sodium Ascorbate | Reducing Agent | 30.00 | 5.00 |
| 10 | Polyvinyl Pyrrolidone | Binder | 25.00 | 4.17 |
| 11 | Magnesium Stearate | Lubricant | 5.25 | 0.88 |

| | | |
|---|---|---|
| Total Weight | 600.00 | 100.00 |

The invention claimed is:

1. A pharmaceutical formulation that facilitates the enhanced dissolution of an alkaline salt of an oral absorption promoter contained therein, wherein the said pharmaceutical formulation comprises (1) a drug in the amount of about 0.01-85% (w/w) of the total amount of the pharmaceutical formulation, (2) an alkaline salt of an oral absorption promoter in the amount of about 5-98% (w/w) of the total amount of the pharmaceutical formulation, (3) an acid neutralizer in an amount of about 3.5-35% (w/w) of the total amount of the pharmaceutical formulation or in an amount that is at least sufficient to elevate the pH of mixture of 35 mL of 0.1N HCl and 125 ml of water from less than about 2 to at least about 2.5, and (4) optionally one or more pharmaceutically acceptable excipients,
wherein the said pharmaceutical formulation, when subjected to an in vitro release test in the 160 mL of acidic medium having a pH of about 1.7, released the acid neutralizer prior to, concurrently with, or faster than the release of the alkaline salt of the oral absorption promoter, which facilitates marked improvement in the release or dissolution of the alkaline salt of the oral absorption promoter in the 160 mL of the acidic medium.

2. The pharmaceutical formulation of claim 1 released at least about 35% of the total amount of the acid neutralizer contained in the pharmaceutical formulation prior to beginning the release of the alkaline salt of the oral absorption promoter.

3. The pharmaceutical formulation of claim 1 released the acid neutralizer faster than the release of the alkaline salt of the oral absorption promoter in first 5 minutes.

4. The pharmaceutical formulation of claim 1 released the acid neutralizer agent concurrently with the release of the alkaline salt of the oral absorption promoter.

5. The pharmaceutical formulation of claim 1, wherein the acid neutralizer is at least one selected from the group of magnesium oxide, meglumine, sodium oxide, sodium hydroxide, sodium bicarbonate, sodium potassium tartrate, bismuth aluminate, bismuth carbonate, bismuth subcarbonate, bismuth subgallate, bismuth subnitrate, potassium citrate, sodium carbonate, potassium bicarbonate, potassium carbonate, calcium carbonate, calcium phosphate, dibasic calcium phosphate, dihydroxyaluminumaminoacetate, dihydroxyaluminum sodium carbonate, glycine, magnesium glycinate, magnesium hydroxide, magnesium carbonate, sodium borate, aluminum oxide, aluminum hydroxide, ammonium carbonate, monoethanolamide, diethanolamine, triethanolamine, potassium hydroxide, calcium hydroxide, sodium phosphate dibasic, trolamine, sodium potassium tartrate, tribasic sodium phosphate, tricalcium phosphate, any combination thereof.

6. The pharmaceutical formulation of claim 1, wherein the alkaline salt of the oral absorption promoter is the alkaline salt of at least one selected from the group consisting of caproic acid, caprylic acid, capric acid, nipecotic acid, butyric acid, propionic acid, hydroxamic acid, succinic acid, nicotinic acid, valeric acid, nonanoic acid, sebacic acid, sebalic acid, heptanoic acid, carboxylic acid, acetic acid, or any derivative thereof.

7. The pharmaceutical formulation of claim 1, wherein the alkaline salt of the oral absorption promoter is sodium N-(8-(2-hydroxybenzoyl)amino) caprylic acid or sodium caprate.

8. The pharmaceutical formulation of claim 1, wherein the drug is GLP-1 agonist.

9. The pharmaceutical formulation of claim 1, wherein the drug is at least one selected from the group of semaglutide, liraglutide, exenatide, lixizenatide, and orforglipron.

10. The pharmaceutical formulation of claim 1 further comprises a pharmaceutically acceptable amount of at least one intestinal enzyme inhibitor that further afford the protection, at least in part, to the drug from proteolytic degradation.

11. The pharmaceutical formulation of claim 10, wherein the intestinal enzyme inhibitor is at least one metal in the form of any or a combination of a salt thereof and a complex thereof, or a combination of (1) at least one metal in the form of any or a combination of a salt thereof and a complex thereof; and (2) at least one reducing agent.

12. The pharmaceutical formulation of claim 11, wherein the at least one metal is selected from the group of vanadium oxide, sodium vanadate, vanadium sulfate, vanadyl sulfate, vanadium biguanide, bis(maltolato) oxavandium, vanadium acetate, vanadyl picolinate, vanadyl citrate, chromium picolinate, chromium polynicotinate, chromium nicotinate, chromium chloride, chromium acetate, manganese gluconate, manganese sulfate, potassium permanganate, manganese chloride, copper chloride, copper acetate, copper sulfate, copper carbonate, copper lysine complex, copper citrate, copper gluconate, zinc sulfate, zinc chloride, zinc acetate, zinc oxide, zinc ascorbate, zinc caprylate, zinc gluconate, zinc stearate, and zinc carbonate.

13. The pharmaceutical formulation of claim 11, wherein the reducing agent is selected from the group of ascorbic acid, sodium ascorbate, reduced glutathione, cysteine, uric acid, a reducing sugar, a reducing monosaccharide, glucose, glyceraldehyde, galactose, a reducing disaccharide, lactose, maltose, mannitol, alpha-tocopherol, vitamin A, alpha-lipoic acid, dihydro-alpha-lipoic acid, a thiol-bearing compound, a thiomer, and any combination thereof.

14. The pharmaceutical formulation of claim 11, wherein the at least one metal is comprises in the amount of greater than about 0.05% (w/w) but less than about 5% (w/w) of the total amount of the pharmaceutical formulation.

15. The pharmaceutical formulation of claim 11, wherein the reducing agent comprises in the amount ranging from about 30 mg to about 250 mg.

16. The pharmaceutical formulation of claim 1 further comprises one or more pharmaceutically acceptable excipients selected from the group of delivery agent, vehicle, filler or diluents, binder, lubricant, glidant, disintegrant, crystallization retarders, acidifying agent, preservative, antioxidant, buffering agent, chelating agent, complexing agents, surfactant agent, emulsifying or solubilizing agents, sweetening agents, wetting agents stabilizing agent, colouring agent, and flavouring agent.

17. The pharmaceutical formulation of claim 1 is formulated in the form of single or multilayer tablet, capsule, a capsule-in-capsule, tablet-in-capsule, lozenge, solution, emulsion, suspension, syrup, elixir, powder and granules for reconstitution, or multi-particulate dosage forms.

18. The pharmaceutical formulation of claim 18, wherein the multi-particulate dosage form is formulated to sprinkle on any liquid medium or formulated to reconstitute using appropriate amount of any liquid medium prior to administration.

19. The pharmaceutical formulation of claim 1 facilitates the enhanced dissolution of an alkaline salt of an oral absorption promoter contained therein when administered in a fasted state of stomach, optionally.

20. The pharmaceutical formulation of claim 19 is administered with 120 mL of water.
